(19) United States
(12) Patent Application Publication
(10) Pub. No.: US 2005/0244490 A1
(43) Pub. Date: Nov. 3, 2005

(54) Dosing methods for beta-D-2',3'-dideoxy-2',3'-didehydro-5-fluorocytidine antiviral therapy

(76) Inventors: Michael Otto, Lilburn, GA (US); Abel de la Rosa, Alpharetta, GA (US); Krishnaswamy Yeleswaram, Landenberg, PA (US)

Correspondence Address: KING & SPALDING LLP, 191 PEACHTREE STREET, N.E., 45TH FLOOR, ATLANTA, GA, 30303-1763, US

(21) Appl. No.: 11/008,693

(22) Filed: Dec. 9, 2004

Related U.S. Application Data
(60) Provisional application No. 60/528,138, filed on Dec. 9, 2003.

(51) Int. Cl. A61K 31/7072; A61K 9/48; A61K 9/24

(52) U.S. Cl. 424/451; 424/472; 514/49

(57) ABSTRACT

The disclosed invention is a composition for and a method of treating a HIV infection in a host, such as a human, using a single, once a day, oral dose of β-D-D4FC in an enteric-coated tablet. The enterically coated β-D-D4FC increases the amount of the drug that remains in active form for use in inhibiting the HIV virus in vivo.

### RELATED APPLICATIONS TO THE INVENTION

This application claims priority to U.S. Provisional Application No. 60/528,138, filed Dec. 9, 2003.

### FIELD OF THE INVENTION

This invention describes dosing strategies for 2′,3′-dideoxy-2′,3′-didehydro-5-fluoro-cytidine antiviral therapies.

### BACKGROUND OF THE INVENTION

In the two decades since its discovery, HIV has become a significant global health problem. The syndrome now known as AIDS (Acquired Immune Deficiency Syndrome) includes more than 25 AIDS associated conditions or diseases. More than 60 million people have been infected with HIV, and currently more than 40 million people are estimated to be living with HIV/AIDS. An estimated 5 million people became infected with HIV in 2003, and more than 95% of these new infections were in developing countries. UNAIDS, AIDS Epidemic Update, December, 2003. The disease is the now the fourth leading cause of death worldwide. AIDS is disproportionately a disease of the young, with more that half of HIV positive individuals infected before the age of 25 and succumbing to disease and ultimately death before the age of 35. As a result, more than 14 million children have been orphaned by AIDS.

Numerous compounds have been synthesized to combat the human immunodeficiency virus (HIV) since it was discovered to be the etiological cause of the acquired immunodeficiency syndrome (AIDS) in 1983. A focal point of AIDS research efforts has been and continues to be the development of inhibitors of human immunodeficiency virus (HIV-1) reverse transcriptase, the enzyme responsible for the reverse transcription of the retroviral RNA to proviral DNA (W. C. Greene, New England Journal of Medicine (1991), 324:308-17; Mitsuya et al., Science (1990), 249:1533-44; E. J. DeClercq, Retrovirus (1992), 8:119-34). Inhibitors include non-nucleoside reverse transcriptase inhibitors or NNRTIs that bind to a specific allosteric site of the HIV reverse transcriptase near the polymerase site and interfere with reverse transcription by either altering the conformation or the mobility of the reverse transcriptase, thus leading to noncompetitive inhibition of the enzyme (Kohlstaedt et al., Science (1992), 256:1783-90).

β-D-2′,3′-Didehydro-2′,3′-dideoxy-5-fluorocytidine (“β-D-D4FC”), which has the structure 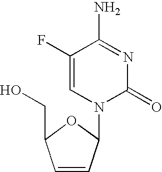
is currently in human clinical trials for the treatment of HIV. The compound exhibits potent anti-HIV activity in vitro. See Schinazi et al., J. Med. Chem. 1999, 42, 859-867.

U.S. Pat. No. 6,232,300 discloses a method for treating a host infected with human immunodeficiency virus comprising administering an effective amount of β-D-2′,3′-dideoxy-2′,3′-didehydro-5-fluorocytidine (D4FC) or a pharmaceutically acceptable salt thereof. U.S. Pat. No. 6,391,859 discloses a method for treating a host infected with human immunodeficiency virus comprising administering an effective amount of a physiologically acceptable ester of β-D-2′,3′-dideoxy-2′,3′-didehydro-5-fluorocytidine (D4FC) or a pharmaceutically acceptable salt thereof. See also U.S. Patent Application Publication No. 2002/0198173.

U.S. Pat. No. 5,905,070 discloses a method for the treatment of HIV and HBV infection that includes administering an effective amount of β-D-D4FC in combination or alternation with cis-2-hydroxymethyl-5-(5-fluorocytosin-1-yl)-1,3-oxathiolane, cis-2-hydroxymethyl-5-(cytosin-1-yl)-1,3-oxathiolane, 9-[4-(hydroxymethyl)-2-cyclopenten-1-yl)-guanine (carbovir), 9-[(2-hydroxyethoxy)methyl]-guanine (acyclovir), interferon, 3′-deoxy-3′-azido-thymidine (AZT), 2′,3′-dideoxyinosine (DDI), 2′,3′-dideoxycytidine (DDC), (−)-2′-fluoro-5-methyl-β-L-ara-uridine (L-FMAU) or 2′,3′-didehydro-2′,3′-dideoxythymidine (D4T).

U.S. Pat. No. 5,703,058 discloses a method for the treatment of HIV and HBV infection that includes administering an effective amount of β-L-D4FC in combination or alternation with cis-2-hydroxymethyl-5-(5-fluorocytosin-1-yl)-1,3-oxathiolane, cis-2-hydroxymethyl-5-(cytosin-1-yl)-1,3-oxathiolane, 9-[4-(hydroxy-methyl)-2-cyclopenten-1-yl)-guanine (carbovir), 9-[(2-hydroxyethoxy)methyl]-guanine (acyclovir), interferon, 3′-deoxy-3′-azido-thymidine (AZT), 2′,3′-dideoxyinosine (DDI), 2′,3′-dideoxycytidine (DDC), (−)-2′-fluoro-5-methyl-β-L-ara-uridine (L-FMAU) or 2′,3′-didehydro-2′,3′-dideoxythymidine (D4T).

European Patent Application Publication No. 0 409 227 A2 filed by Ajinomoto Co., Inc., discloses β-D-D4FC (Example 2) and its use to treat hepatitis B.

Netherlands Patent No. 8901258 filed by Stichting Rega V. Z. W. discloses generally 5-halogeno-2′,3′-dideoxy-2′,3′-didehydrocytidine derivatives for use in treating HIV and hepatitis B (“HBV”).

International Publication No. WO 00/43014 discloses methods for treating HIV that includes administering β-D-D4FC or its pharmaceutically acceptable salt or prodrug to a human in need of therapy in combination or alternation with a drug that induces a mutation in HIV-1 at a location other than the 70(K to N), 90 or the 172 codons of the reverse transcriptase region. Also disclosed is a method for using β-D-D4FC as “salvage therapy” to patients which exhibit drug resistance to other anti-HIV agents. β-D-D4FC can be used generally as salvage therapy for any patient which exhibits resistance to a drug that induces a mutation at other than the 70(K to N), 90 or the 172 codons.

In Dec. 15, 2002 and Jun. 10, 2003, Pharmasset presented data regarding the pharmacokinetics of enteric-coated β-D-D4FC when administered as a single oral dose to HIV-1 infected males. R. L. Murphy, et al. “Pharmacokinetics and Safety of the Nucleoside Reverset in HIV-1 Infected Patients” HIV-DART 2002 Frontiers in Drug Development for Antiretroviral Therapies, Naples, Fl., Dec. 15-19, 2002; L. Stuyver, et al. “Antiviral Activity of the Nucleoside Reverset Following Single Oral Doses in Hiv-1 Infected Patients” XII International HIV Drug Resistance Workshop: Basic Principles and Clinical Implications, Jun. 10-14, 2003, Los Cabos, Mexico.

β-D-2′,3′-didehydro-2′,3′-dideoxy-5-fluorocytidine (β-D-D4FC, Reverset™, RVT, DPC-817) retains activity against 3TC and AZT-resistant HIV-1 in vitro. Schinazi, R. F. et al. “DPC 817: A cytidine nucleoside analog with activity against zudovudine- and lamivudine-resistant viral variants” Antimicrob. Agents Chemother. 2002, 46, 1394-1401; Geleziunas, R. et al. “HIV-1 resistance profile of the novel nucleoside reverse transcriptase inhibitor D-d4FC” Antiviral Chemistry & Chemotherapy, 2003, 14, 49-59.

Several syntheses for the preparation of β-D-D4FC or its enantiomer, β-L-D4FC, have been reported. See Schinazi R. F. et al, J. Med. Chem. 1999, 42,859-867; Chen S., Biorganic & Medicinal Chemistry Letters 8 (1998) 3245-3250; Doyle, T. W., et al., J. Org. Chem. 1997, 62, 3449-3452; Cheng, Y., et al., J. Med. Chem. 1996, 62, 1757-1759; and Lin et al., U.S. Pat. No. 5,561,120.

While β-D-D4FC has been shown to have potent anti-HIV activity, it is a goal to enhance its activity and usefulness in vivo.

Therefore, it is an object of the present invention to provide compositions and methods which enhance the activity and/or usefulness of β-D-D4FC as an anti-HIV agent for humans.

### SUMMARY OF THE INVENTION

It was surprisingly discovered that when β-D-D4FC is delivered as an enteric-coated tablet, an increased amount of the drug remains in active form for use in inhibiting the HIV virus in vivo.

Specifically, during the clinical development of β-D-2′,3′-didehydro-2′,3′-dideoxy-5-fluorocytidine (β-D-D4FC, Reverse™, RVT) under an approved US FDA Investigational New Drug (IND) application for the treatment of HIV-1 in nucleoside reverse transcriptase inhibitor (NRTI)-experienced patients, it was surprisingly discovered that when β-D-D4FC is delivered as an enteric-coated tablet, the levels of 5FC, which was determined to be a major metabolite of β-D-D4FC, is significantly reduced compared to administration via a buffered solution. There was no 5FU observed in the plasma at doses as high as 200 mg. Therefore, in one embodiment, the present invention is directed to the treatment of HIV in a host comprising administering an effective dose of β-D-D4FC as an enteric-coated tablet, including but not limited to a tablet made via a wet granulation process. In another particular embodiment, the enteric-coated delivery form is in the form of enteric coating on beads or beadlets in a capsule, for example enteric coating on microbeads or microbeadlets in a capsule. In a further embodiment, the enteric-coated tablet are administered to a patient that has fasted.

It also was surprisingly discovered that there was strong, statistically significant viral load drop after only a single oral dose of β-D-D4FC. A single oral dose of β-D-D4FC reduced the viral load by a mean of 0.4±0.2 log₁₀ (approximately 40%) at various dosage levels 24 and 48 hours after administration. The antiviral response over a 24 or 48 hour period was not dose dependent, possibly due to the long intracellular half-life of β-D-D4FC-TP. The Cₘₐₓ and AUC are linear, but not proportional with dose. A mean Cₘₐₓ of 2.5 μM can be achieved with a 50 mg dose. At 200 mg, the mean Cₘₐₓ remains above 5 μM for ≧3.5 hours. The plasma levels remain above the median effective concentrations for β-D-D4FC in human PBM cells for >24 hours. Since β-D-D4FC has high oral bioavailability and low pill burden, β-D-D4FC can be useful as a once-a-day treatment for HIV.

In one embodiment of the invention, the single oral dose of β-D-D4FC is an effective dose that achieves a plasma level of at least around 5, 6, 7, or 10 μM a day. In a particular embodiment of the present invention, the single oral dose of β-D-D4FC is 25 mg a day. In another particular embodiment of the present invention, the single oral dose of β-D-D4FC is 50 mg a day. In another particular embodiment of the present invention, the single oral dose of β-D-D4FC is 100 mg a day. In yet another particular embodiment of the present invention, the single oral dose of β-D-D4FC is 200 or 250 mg a day. In one embodiment of the present invention, the single oral dose of β-D-D4FC is from around 25 mg a day to around 250 mg a day.

Based on the in vitro potency of β-D-D4FC against both wild type and NRTI-resistant HIV-1, the favorable PK values, as well as the in vivo activity observed after only a single dose of drug, β-D-D4FC can be useful as a once-a-day component of treatment regimens for NRTI-experienced and treatment-naïve patients.

In a further embodiment of the present invention, methods for the treatment of an HIV infection is provided comprising administering an effective amount of β-D-D4FC in an enteric-coated tablet for once a day treatment. In another embodiment of the present invention, methods for the treatment of an HIV infection is provided comprising administering an effective amount of β-D-D4FC in an enteric-coated tablet for once a day treatment for at least 10 days. In a particular embodiment of the present invention, the effective amount of β-D-D4FC for once a day treatment is a dosage that achieves a plasma level of at least around 2.5 μM, such as between around 25 mg to about 250 mg a day, and in particular 25 mg, 50 mg, 75, mg, 100 mg, 150 mg, 200 mg, or 250 mg a day.

### BRIEF DESCRIPTION OF THE FIGURES

FIG. 1 is a schematic that depicts the potential degradation of β-D-D4FC to 5-fluorocytosine (5FC) and 5-fluorouracil (5FU).

FIG. 2 is a graphical representation of the pharmacokinetic analysis for β-D-D4FC, which illustrates the mean plasma concentrations of β-D-D4FC after a single oral dose. The relationship between the Cₘₐₓ and the AUC values was studied for 10, 25, and 50 mg in buffered solutions (sol), and 50, 100, and 200 mg as enteric-coated tablets (tab); 6 subjects per group. The relationship between Cₘₐₓ and AUC was linear for both solution and tablet.

FIG. 3 is a graphical representation of the pharmacokinetic analysis for 5FC, which illustrates the mean plasma concentrations of 5FC after a single oral dose. The relationship between the Cₘₐₓ, and the AUC values was determined for 5FC after doses of β-D-D4FC at 10, 25, and 50 mg in buffered solutions (sol), and 50, 100, and 200 mg as enteric-coated tablets (tab); 6 subjects per group. The relationship between Cₘₐₓ and AUC was linear for both solution and tablet. Enteric coating resulted in a 3-fold reduction in the amount of 5FC in the plasma. No 5FU was detected in the plasma at any dose.

FIG. 4 depicts the plasma HIV-1 viral load changes in exemplary viral load profiles of patients in each cohort. (A) Subject 106—representative for Cohort 1—a 10 mg dose administration was followed by a placebo administration, followed by a 25 mg dose. (B) Subject 204—representative for Cohort 2—a 50 mg dose administration in buffered solution was followed by a placebo administration, followed by a 50 mg tablet. (C) Subject 306—representative for Cohort 3—a 100 mg dose administration was followed by a placebo administration, followed by a 200 mg dose. (D) Summary of viral load for all subjects in each cohort.

FIG. 5 is a line graph that depicts the mean change in viral load (in log₁₀) as compared to the administered dose of β-D-D4FC.

FIG. 6 is a line graph that depicts the relationship between antiviral effect and the amount of β-D-D4FC exposure by plotting viral load versus mean Cₘₐₓ values.

FIG. 7 is a bar graph that depicts the antiviral activity of β-D-D4FC against mutant strains of HIV in vitro. FIG. 7b illustrates the Virco profiling against recombinant clinical isolates of β-D-D4FC versus other nucleoside analogs. A panel of 22 viruses was constructed in the HXB2 background using the Protease and RT sequences from clinical isolates. Viruses contained 2 to 17 mutations in RT frequently associated with nucleoside resistance, including: M184V, M41L, D67N, T215Y.

FIG. 8 are line graphs that illustrate the uptake and conversion of β-D-D4FC to β-D-D4FC-TP.

FIG. 9 is a line graph that illustrates the determination of intra-cellular half-life. Time samples were analyzed for remaining β-D-D4FC-TP content.

FIG. 10 is a bar graph that represents the functional half-life of β-D-D4FC as compared to 3TC after 24 hours.

FIG. 11 is a bar graph that illustrates the differences in inhibition 2 versus 24 hours after exposure to β-D-D4FC, indicating that brief exposure to β-D-D4FC is sufficient for antiviral activity.

FIG. 12a is a line graph that indicates the log copies of plasma HIV RNA following 10-day mono-therapy with 50 mg of β-D-D4FC. FIG. 12b is a line graph that indicates the log change in HIV RNA following 10-day mono-therapy with 50 mg of β-D-D4FC.

FIG. 13 is a line graph that illustrates the mean blood plasma concentrations (and standard deviations) over time for the fed (dotted line) and fasted (solid line) regimen.

FIG. 14 is a bar graph that depicts the change in CD4 counts after administration of a placebo and a single 50 mg dose of β-D-D4FC after 10 and 21 days.

FIG. 15 is a line graph that illustrates the mean percent change in plasma HIV RNA after a single dose of β-D-D4FC.

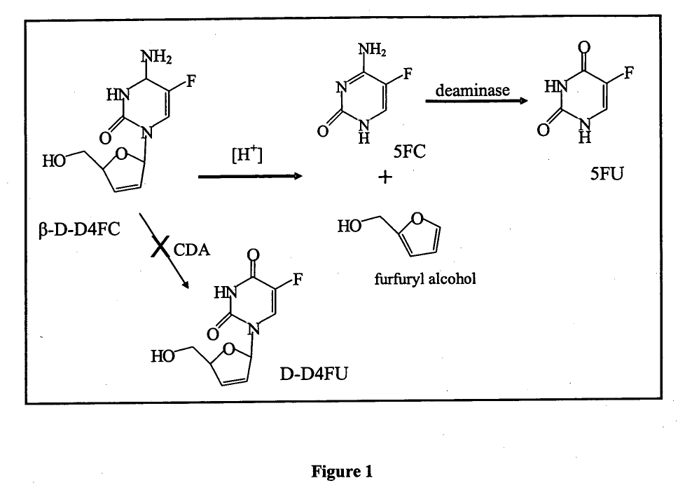

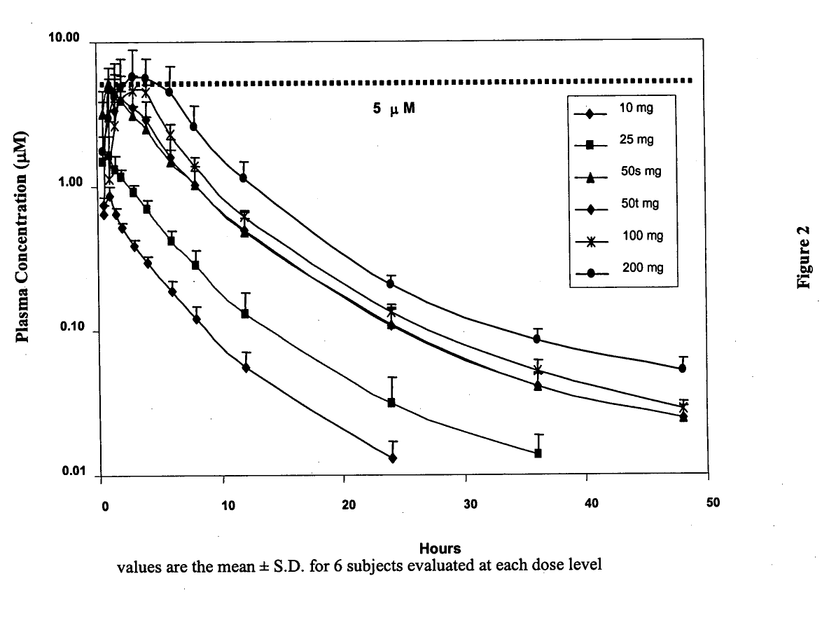

### DETAILED DESCRIPTION OF THE INVENTION

It was surprisingly discovered that when β-D-D4FC is delivered as an enteric-coated tablet, an increased amount of the drug remains in active form for use in inhibiting the HIV virus in vivo.

Specifically, during the clinical development of β-D-2′,3′-didehydro-2′,3′-dideoxy-5-fluorocytidine (β-D-D4FC, Reverset™, RVT) under an approved US FDA Investigational New Drug (IND) application for the treatment of HIV-1 in nucleoside reverse transcriptase inhibitor (NRTI)-experienced patients, it was surprisingly discovered that when β-D-D4FC is delivered as an enteric-coated tablet, the levels of 5FC, which was determined to be a major metabolite of β-D-D4FC, is significantly reduced compared to administration via a buffered solution. There was no 5FU observed in the plasma at doses as high as 200 mg. Therefore, in one embodiment, the present invention is directed to the treatment of HIV in a host comprising administering an effective dose of β-D-D4FC as an enteric-coated tablet, including but not limited to a tablet made via a wet granulation process. In another particular embodiment, the enteric-coated delivery form is in the form of enteric coating on beads or beadlets in a capsule, for example enteric coating on microbeads or microbeadlets in a capsule. In a further embodiment, the enteric-coated tablet are administered to a patient that has fasted.

It also was surprisingly discovered that there was strong, statistically significant viral load drop after only a single oral dose of β-D-D4FC. A single oral dose of β-D-D4FC reduced the viral load by a mean of 0.4±0.2 log₁₀ (approximately 40%) at various dosage levels 24 and 48 hours after administration. The antiviral response over a 24 or 48 hour period was not dose dependent, possibly due to the long intracellular half-life of β-D-D4FC-TP. The Cₘₐₓ and AUC are linear, but not proportional with dose. A mean Cₘₐₓ of 2.5 μM can be achieved with a 50 mg dose. At 200 mg, the mean Cₘₐₓ remains above 5 μM for ≧3.5 hours. The plasma levels remain above the median effective concentrations for β-D-D4FC in human PBM cells for >24 hours. Since β-D-D4FC has high oral bioavailability and low pill burden, β-D-D4FC can be useful as a once-a-day treatment for HIV.

In one embodiment of the invention, the single oral dose of β-D-D4FC is an effective dose that achieves a plasma level of at least around 5, 6, 7, or 10 μM a day. In a particular embodiment of the present invention, the single oral dose of β-D-D4FC is 25 mg a day. In another particular embodiment of the present invention, the single oral dose of β-D-D4FC is 50 mg a day. In another particular embodiment of the present invention, the single oral dose of β-D-D4FC is 100 mg a day. In yet another particular embodiment of the present invention, the single oral dose of β-D-D4FC is 200 or 250 mg a day. In one embodiment of the present invention, the single oral dose of β-D-D4FC is from around 25 mg a day to around 250 mg a day.

Based on the in vitro potency of β-D-D4FC against both wild type and NRTI-resistant HIV-1, the favorable PK values, as well as the in vivo activity observed after only a single dose of drug, β-D-D4FC can be useful as a once-a-day component of treatment regimens for NRTI-experienced and treatment-naïve patients.

In a further embodiment of the present invention, methods for the treatment of an HIV infection is provided comprising administering an effective amount of β-D-D4FC in an enteric-coated tablet for once a day treatment. In another embodiment of the present invention, methods for the treatment of an HIV infection is provided comprising administering an effective amount of β-D-D4FC in an enteric-coated tablet for once a day treatment for at least 10 days. In a particular embodiment of the present invention, the effective amount of β-D-D4FC for once a day treatment is a dosage that achieves a plasma level of at least around 2.5 μM, such as between around 25 mg to about 250 mg a day, and in particular 25 mg, 50 mg, 75, mg, 100 mg, 150 mg, 200 mg, or 250 mg a day.

The active compound can be administered as any salt or prodrug that upon administration to the recipient is capable of providing directly or indirectly the parent compound, or that exhibits activity itself. Nonlimiting examples are the pharmaceutically acceptable salts (alternatively referred to as “physiologically acceptable salts”). Further, the modifications can affect the biological activity of the compound, in some cases increasing the activity over the parent compound. This can easily be assessed by preparing the salt or prodrug and testing its antiviral activity according to the methods described herein, or other methods known to those skilled in the art.

As used herein, the term pharmaceutically acceptable salts refers to salts that retain the desired biological activity of the herein-identified compounds and exhibit minimal undesired toxicological effects. Non-limiting examples of such salts are base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, ethylene-diamine, or the like.

Definitions

As used herein, the term “substantially free of;” “substantially in the absence of” or “isolated” refers to a nucleoside composition that includes at least 95%, and preferably 99% to 100% by weight, of the designated enantiomer of that nucleoside. In a preferred embodiment, the process produces compounds that are substantially free of enantiomers of the opposite configuration.

The term “enantiomerically enriched” is used throughout the specification to describe a nucleoside which includes at least about 95%, preferably at least 96%, more preferably at least 97%, even more preferably, at least 98%, and even more preferably at least about 99% or more of a single enantiomer of that nucleoside. When a nucleoside of a particular configuration (D or L) is referred to in this specification, it is presumed that the nucleoside is an enantiomerically enriched nucleoside, unless otherwise stated.

The term “administered with food” or “administered with a meal” includes administering the drug at the meal, or within 2 hours, 1 hour, or 30 minutes after the meal.

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention either possess antiviral activity against HIV, or are metabolized to a compound that exhibits such activity.

Pharmaceutically Acceptable Salts, Esters, and Prodrugs

Pharmaceutically acceptable salts include those derived from pharmaceutically acceptable inorganic or organic bases and acids. Non-limiting examples of such salts are (a) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine; (b) acid addition salts formed with inorganic acids (for example, hydrochloric acid, hydrobromic acid, sulfuric acid, phosphoric acid, nitric acid, and the like), and salts formed with organic acids such as amino acid, acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, and polygalacturonic acid, which form a physiological acceptable anion, for example, tosylate, methanesulfonate, acetate, citrate, malonate, tartarate, succinate, benzoate, ascorbate, α-ketoglutarate, and α-glycerophosphate; or (c) combinations of (a) and (b); e.g., a zinc tannate salt or the like. Suitable inorganic salts may also be formed, including, sulfate, nitrate, bicarbonate, and carbonate salts. In one embodiment, the pharmaceutically acceptable salt is not an acid addition salt. In one embodiment, the pharmaceutically acceptable salt is a base addition salt.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the hydroxyl group of the compound or of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

Any of the nucleosides which are described herein can be administered as an acylated prodrug, wherein the term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl).

The active nucleoside or other hydroxyl containing compound can also be provided as an ether lipid (and particularly a 5′-ether lipid or a 5′-phosphoether lipid for a nucleoside), as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hostetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside or other hydroxyl or amine containing compound, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Nonlimiting examples of nucleotide prodrugs are described in the following references: Ho, D. H. W. (1973) “Distribution of Kinase and deaminase of 1-β-D-arabinofuranosylcytosine in tissues of man and muse.” Cancer Res. 33, 2816-2820; Holy, A. (1993) Isopolar phosphorous-modified nucleotide analogues,” In: De Clercq (Ed.), Advances in Antiviral Drug Design, Vol. I, JAI Press, pp. 179-231; Hong, C. I., Nechaev, A., and West, C. R. (1979a) “Synthesis and antitumor activity of 1-β-D-arabino-furanosylcytosine conjugates of cortisol and cortisone.” Bicohem. Biophys. Rs. Commun. 88, 1223-1229; Hong, C. I., Nechaev, A., Kirisits, A. J. Buchheit, D. J. and West, C. R. (1980) “Nucleoside conjugates as potential antitumor agents. 3. Synthesis and antitumor activity of 1-(β-D-arabinofuranosyl) cytosine conjugates of corticosteriods and selected lipophilic alcohols.” J. Med. Chem. 28, 171-177; Hosteller, K. Y., Stuhmiller, L. M., Lenting, H. B. M. van den Bosch, H. and Richman J. Biol. Chem. 265, 6112-6117; Hosteller, K. Y., Carson, D. A. and Richman, D. D. (1991); “Phosphatidylazidothymidine: mechanism of antiretroviral action in CEM cells.” J. Biol. Chem. 266, 11714-11717; Hosteller, K. Y., Korba, B. Sridhar, C., Gardener, M. (1994a) “Antiviral activity of phosphatidyl-dideoxycytidine in hepatitis B-infected cells and enhanced hepatic uptake in mice.” Antiviral Res. 24, 59-67; Hosteller, K. Y., Richman, D. D., Sridhar. C. N. Felgner, P. L. Felgner, J., Ricci, J., Gardener, M. F. Selleseth, D. W. and Ellis, M. N. (1994b) “Phosphatidylazidothymidine and phosphatidyl-ddC: Assessment of uptake in mouse lymphoid tissues and antiviral activities in human immunodeficiency virus-infected cells and in rauscher leukemia virus-infected mice.” Antimicrobial Agents Chemother. 38, 2792-2797; Hunston, R. N., Jones, A. A. McGuigan, C., Walker, R. T., Balzarini, J., and DeClercq, E. (1984) “Synthesis and biological properties of some cyclic phosphotriesters derived from 2′-deoxy-5-fluorouridine.” J. Med. Chem. 27, 440-444; Ji, Y. H., Moog, C., Schmitt, G., Bischoff, P. and Luu, B. (1990); “Monophosphoric acid esters of 7-β-hydroxycholesterol and of pyrimidine nucleoside as potential antitumor agents: synthesis and preliminary evaluation of antitumor activity.” J. Med. Chem. 33 2264-2270; Jones, A. S., McGuigan, C., Walker, R. T., Balzarini, J. and DeClercq, E. (1984) “Synthesis, properties, and biological activity of some nucleoside cyclic phosphoramidates.” J. Chem. Soc. Perkin Trans. I, 1471-1474; Juodka, B. A. and Smrt, J. (1974) “Synthesis of diribonucleoside phosph (P®N) amino acid derivatives.” Coll. Czech. Chem. Comm. 39, 363-968; Kataoka, S., Imai, J., Yamaji, N., Kato, M., Saito, M., Kawada, T. and Imai, S. (1989) “Alkylated cAMP derivatives; selective synthesis and biological activities.” Nucleic Acids Res. Sym. Ser. 21, 1-2; Kataoka, S., Uchida, “(cAMP) benzyl and methyl triesters.” Heterocycles 32, 1351-1356; Kinchington, D., Harvey, J. J., O'Connor, T. J., Jones, B. C. N. M., Devine, K. G., Taylor-Robinson D., Jeffries, D. J. and McGuigan, C. (1992) “Comparison of antiviral effects of zidovudine phosphoramidate and phosphorodiamidate derivatives against HIV and ULV in vitro.” Antiviral Chem. Chemother. 3, 107-112; Kodama, K., Morozumi, M., Saithoh, K. I., Kuninaka, H., Yosino, H. and Saneyoshi, M. (1989) “Antitumor activity and pharmacology of 1-β-D-arabinofuranosylcytosine-5′-stearylphosphate; an orally active derivative of 1-β-D-arabinofuranosylcytosine.” Jpn. J. Cancer Res. 80, 679-685; Korty, M. and Engels, J. (1979) “The effects of adenosine- and guanosine 3′,5′ phosphoric and acid benzyl esters on guinea-pig ventricular myocardium.” Naunyn-Schmiedeberg's Arch. Pharmacol. 310, 103-111; Kumar, A., Goe, P. L., Jones, A. S. Walker, R. T. Balzarini, J. and DeClercq, E. (1990) “Synthesis and biological evaluation of some cyclic phosphoramidate nucleoside derivatives.” J. Med. Chem, 33, 2368-2375; LeBec, C., and Huynh-Dinh, T. (1991) “Synthesis of lipophilic phosphate triester derivatives of 5-fluorouridine an arabinocytidine as anticancer prodrugs.” Tetrahedron Lett. 32, 6553-6556; Lichtenstein, J., Barner, H. D. and Cohen, S. S. (1960) “The metabolism of exogenously supplied nucleotides by Escherichia coli.,” J. Biol. Chem. 235, 457-465; Luethy, J., Von Daeniken, A., Friederich, J. Manthey, B., Zweifel, J., Schlatter, C. and Benn, M. H. (1981) “Synthesis and toxicological properties of three naturally occurring cyanoepithioalkanes”. Mitt. Geg. Lebensmittelunters. Hyg. 72, 131-133 (Chem. Abstr. 95, 127093); McGigan, C. Tollerfield, S. M. and Riley, P. a. (1989) “Synthesis and biological evaluation of some phosphate triester derivatives of the anti-viral drug Ara.” Nucleic Acids Res. 17, 6065-6075; McGuigan, C., Devine, K. G., O'Connor, T. J., Galpin, S. A., Jeffries, D. J. and Kinchington, D. (1990a) “Synthesis and evaluation of some novel phosphoramidate derivatives of 3′-azido-3′-deoxythymidine (AZT) as anti-HIV compounds.” Antiviral Chem. Chemother. 1 107-113; McGuigan, C., O'Connor, T. J., Nicholls, S. R. Nickson, C. and Kinchington, D. (1990b) “Synthesis and anti-HIV activity of some novel substituted dialkyl phosphate derivatives of AZT and ddCyd.” Antiviral Chem. Chemother. 1, 355-360; McGuigan, C., Nicholls, S. R., O'Connor, T. J., and Kinchington, D. (1990c) “Synthesis of some novel dialkyl phosphate derivative of 3′-modified nucleosides as potential anti-AIDS drugs.” Antiviral Chem. Chemother. 1, 25-33; McGuigan, C., Devin, K. G., O'Connor, T. J., and Kinchington, D. (1991) “Synthesis and anti-HIV activity of some haloalkyl phosphoramidate derivatives of 3′-azido-3′ deoxythymidine (AZT); potent activity of the trichloroethyl methoxyalaninyl compound.” Antiviral Res. 15, 255-263; McGuigan, C., Pathirana, R. N., Balzarini, J. and DeClercq, E. (1993b) “Intracellular delivery of bioactive AZT nucleotides by aryl phosphate derivatives of AZT.” J. Med. Chem. 36, 1048-1052.

Alkyl hydrogen phosphate derivatives of the anti-HIV agent AZT may be less toxic than the parent nucleoside analogue. Antiviral Chem. Chemother. 5, 271-277; Meyer, R. B., Jr., Shuman, D. A. and Robins, R. K. (1973) “Synthesis of purine nucleoside 3′,5′-cyclic phosphoramidates.” Tetrahedron Lett. 269-272; Nagyvary, J. Gohil, R. N., Kirchner, C. R. and Stevens, J. D. (1973) “Studies on neutral esters of cyclic AMP,” BioChem. Biophys. Res. Commun. 55, 1072-1077; Namane, A. Gouyette, C., Fillion, M. P., Fillion, G. and Huynh-Dinh, T. (1992) “Improved brain delivery of AZT using a glycosyl phosphotriester prodrug.” J. Med. Chem. 35, 3039-3044; Nargeot, J. Nerbonne, J. M. Engels, J. and Leser, H. A. (1983) Natl. Acad. Sci. U.S.A. 80, 2395-2399; Nelson, K. A., Bentrude, W. G. Stser, W. N. and Hutchinson, J. P. (1987) “The question of chair-twist equilibria for the phosphate rings of nucleoside cyclic 3′, 5′ monophosphates. 1HNMR and x-ray crystallographic study of the diastereomers of thymidine phenyl cyclic 3′,5′-monophosphate.” J. Am. Chem. Soc. 109, 4058-4064; Nerbonne, J. M., Richard, S., Nargeot, J. and Lester, H. A. (1984) “New photoactivatable cyclic nucleotides produce intracellular jumps in cyclic AMP and cyclic GMP concentrations.” Nature 301, 74-76; Neumann, J. M., Herv_, M., Debouzy, J. C., Guerra, F. I., Gouyette, C., Dupraz, B. and Huyny-Dinh, T. (1989) “Synthesis and transmembrane transport studies by NMR of a glycosyl, phospholipid of thymidine.” J. Am. Chem. Soc. 111, 4270-4277; Ohno, R., Tatsumi, N., Hirano, M., Imai, K. Mizoguchi, H., Nakamura, T., Kosaka, M., Takatuski, K., Yamaya, T., Toyama K., Yoshida, T., Masaoka, T., Hashimoto, S., Ohshima, T., Kimura, I., Yamada, K. and Kimura, J. (1991) “Treatment of myelodysplastic syndromes with orally administered 1-β-D-arabinouranosylcytosine-5′ stearylphosphate.” Oncology 48, 451-455. Palomino, E., Kessle, D. and Horwitz, J. P. (1989) “A dihydropyridine carrier system for sustained delivery of 2′,3′dideoxynucleosides to the brain.” J. Med. Chem. 32, 22-625; Perkins, R. M., Barney, S. Wittrock, R., Clark, P. H., Levin, R. Lambert, D. M., Petteway, S. R., Serafinowska, H. T., Bailey, S. M., Jackson, S., Harnden, M. R. Ashton, R., Sutton, D., Harvey, J. J. and Brown, A. G. (1993) “Activity of BRL47923 and its oral prodrug, SB203657A against a rauscher murine leukemia virus infection in mice.” Antiviral Res. 20 (Suppl. I). 84; Piantadosi, C., Marasco, C. J., Jr., Norris-Natschke, S. L., Meyer, K. L., Gumus, F., Surles, J. R., Ishaq, K. S., Kucera, L. S. Iyer, N., Wallen, C. A., Piantadosi, S. and Modest, E. J. (1991) “Synthesis and evaluation of novel ether lipid nucleoside conjugates for anti-HIV-1 activity.” J. Med. Chem. 34, 1408-1414; Pompon, A., Lefebvre, I., Imbach, J. L., Kahn, S. and Farquhar, D. (1994). “Decomposition pathways of the mono- and bis(pivaloyloxymethyl)esters of azidothymidine-5′-monophosphate in cell extract and in tissue culture medium; an application of the ‘on-line ISRP-cleaning HPLC technique.” Antiviral Chem Chemother. 5, 91-98; Postemark, T. (1974) “Cyclic AMP and cyclic GMP.” Annu. Rev. Pharmacol. 14, 23-33; Prisbe, E. J., Martin, J. C. M., McGhee, D. P. C., Barker, M. F., Smee, D. F. Duke, A. E., Matthews, T. R. and Verheyden, J. P. J. (1986) “Synthesis and antiherpes virus activity of phosphate an phosphonate derivatives of 9-[(1,3-dihydroxy-2-propoxy)methyl]guanine.” J. Med. Chem. 29, 671-675; Pucch, F., Gosselin, G., Lefebvre, I., Pompon, a., Aubertin, A. M. Dim, and Imbach, J. L. (1993) “Intracellular delivery of nucleoside monophosphate through a reductase-mediated activation process.” Antiviral Res. 22, 155-174; Pugaeva, V. P., Klochkeva, S. I., Mashbits, F. D. and Eizengart, R. S. (1969). “Toxicological assessment and health standard ratings for ethylene sulfide in the industrial atmosphere.” Gig. Trf. Prof. Zabol. 14, 47-48 (Chem. Abstr. 72, 212); Robins, R. K. (1984) “The potential of nucleotide analogs as inhibitors of Retro viruses and tumors.” Pharm. Res. 11-18; Rosowsky, A., Kim. S. H., Ross and J. Wick, M. M. (1982) “Lipophilic 5′-(alkylphosphate)esters of 1-β-D-arabinofuranosylcytosine and its N4-acyl and 2.2′-anhydro-3′-O-acyl derivatives as potential prodrugs.” J. Med. Chem. 25, 171-178; Ross, W. (1961) “Increased sensitivity of the walker turnout towards aromatic nitrogen mustards carrying basic side chains following glucose pretreatment.” BioChem. Pharm. 8, 235-240; Ryu, E. K., Ross, R. J. Matsushita, T., MacCoss, M., Hong, C. I. and West, C. R. (1982). “Phospholipid-nucleoside conjugates. 3. Synthesis and preliminary biological evaluation of 1-β-D-arabinofuranosylcytosine 5′ diphosphate [−], 2-diacylglycerols.” J. Med. Chem. 25, 1322-1329; Saffhill, R. and Hume, W. J. (1986) “The degradation of 5-iododeoxyuridine and 5-bromoethoxyuridine by serum from different sources and its consequences for the use of these compounds for incorporation into DNA.” Chem. Biol. Interact. 57, 347-355; Saneyoshi, M., Morozumi, M., Kodama, K., Machida, J., Kuninaka, A. and Yoshino, H. (1980) “Synthetic nucleosides and nucleotides. XVI. Synthesis and biological evaluations of a series of 1-β-D-arabinofuranosylcytosine 5′-alkyl or arylphosphates.” Chem Pharm. Bull. 28, 2915-2923; Sastry, J. K., Nehete, P. N., Khan, S., Nowak, B. J., Plunkett, W., Arlinghaus, R. B. and Farquhar, D. (1992) “Membrane-permeable dideoxyuridine 5′-monophosphate analogue inhibits human immunodeficiency virus infection.” Mol. Pharmacol. 41, 441-445; Shaw, J. P., Jones, R. J. Arimilli, M. N., Louie, M. S., Lee, W. A. and Cundy, K. C. (1994) “Oral bioavailability of PMEA from PMEA prodrugs in male Sprague-Dawley rats.” 9th Annual AAPS Meeting. San Diego, Calif. (Abstract). Shuto, S., Ueda, S., Imamura, S., Fukukawa, K. Matsuda, A. and Ueda, T. (1987) “A facile one-step synthesis of 5′ phosphatidiylnucleosides by an enzymatic two-phase reaction.” Tetrahedron Lett. 28, 199-202; Shuto, S. Itoh, H., Ueda, S., Imamura, S., Kukukawa, K., Tsujino, M., Matsuda, A. and Ueda, T. (1988) Pharm. Bull. 36, 209-217. An example of a useful phosphate prodrug group is the S-acyl-2-thioethyl group, also referred to as “SATE”.

Enteric Formulations

An enteric coating is a coating on a material that protects the material, for example from acidic environments, until it reaches the small intestine. The enteric coating of the present invention can be any enteric composition that is known in the art. The enteric coated β-D-D4FC dosage form can be prepared in any manner known to those skilled in the art, including, as non-limiting embodiments, any of the methods described herein. One β-D-D4FC enteric formulation is a tablet formulation comprising a) a core comprising β-D-D4FC, optionally layered on a seed/sphere, optionally comprising a hydrophilic or hydrophobic matrix containing β-D-D4FC, optionally with one or more other active agent, and optionally pharmaceutically acceptable excipients, b) an optional separating layer; c) an enteric layer and a pharmaceutically acceptable excipient; d) an optional finishing layer.

Core

In one embodiment, the active agent, after optionally mixing with an alkaline compound, is mixed with suitable constituents including a binding agent and formulated into a core material. Said core materials may be produced by extrusion/spheronization, balling or compression and by utilizing different process equipment. The manufactured core materials can be layered further with additional ingredients, optionally comprising active substance, and/or be used for further processing.

Alternatively, inert seeds are layered with active substance (the active substance is optionally mixed with alkaline compounds) can be used as the core material for the further processing. The seeds, which are to be layered with the active substance, can be water insoluble seeds comprising different oxides, celluloses, organic polymers and other materials, alone or in mixtures or water soluble seeds comprising different inorganic salts, sugars, non-pareils and other materials, alone or in mixtures.

Before the seeds are layered, for instance by using granulating or spray coating/layering equipment, the active agent is mixed with a binding agent and optionally further components. Such further components can be binders, surfactants, fillers, disintegrating agents, alkaline additives or other pharmaceutically acceptable ingredients, alone or in mixtures.

In particular, because β-D-D4FC is an acid labile compound, the core materials can be formulated with an alkaline, though otherwise inert, pharmaceutically acceptable substance or substances. In one embodiment, the alkaline substance(s) creates a “micro-pH” around the active compound of not less than pH-7, preferably not less than pH=8, when water is adsorbed to the particles of the mixture or when water is added in small amounts to the mixture. Such substances can be chosen among, but are not restricted to substances such as the sodium, potassium, calcium, magnesium and aluminum salts of phosphoric acid, carbonic acid, citric acid or other suitable weak inorganic or organic acids; substances normally used in antacid preparations such as aluminum, calcium and magnesium hydroxides; magnesium oxide, titanium oxide, or composite substances such as Al₂O₃.MgO.CO₂(Mg₆Al₂(OH)₁₆CO₃.4H₂O), MgO.Al₂O₃.2SiO₂.nH₂O, or similar compounds; organic pH-buffering substances such as trishydroxymethylaminomethane or other similar, pharmaceutically acceptable pH-buffering substances. The stabilizing, high pH-value in the powder mixture can also be achieved by using an alkaline reacting, salt of the active compound such as the sodium, potassium, magnesium, calcium etc. salts of acid labile compounds, either alone or in combination with a conventional buffering substance as previously described.

The core material for the individually enteric coating layered pellets can be composed and formulated according to different principles, such as described in EP 247 983 and WO 96/01623. For instance, the active agent is mixed with one or more pharmaceutical constituents to obtain preferred handling and processing properties and also to obtain a suitable concentration of the active agent in the final mixture. Pharmaceutical constituents such as fillers, binders, lubricants, disintegrating agents, surfactants and other pharmaceutically acceptable additives, can be used.

Optional Separating Layer

The cores containing β-D-D4FC can be optionally separated from the enteric coating polymer(s), in particular when the enteric coating contains free carboxyl groups, which can cause degradation and/or discoloration of the active compound during the coating process of during storage. The separating layer, also referred to as the subcoating layer herein, may also serve as a pH-buffering zone, for example, such that hydrogen ions diffusing from the outside in towards an optionally alkaline core can react with hydroxyl ions diffusing from the alkaline core towards the surface of the coated articles. The pH-buffering properties of the separating layer can be further strengthened by introducing in the layer substances chosen from a group of compounds usually used in antacid formulations such as, for instance, magnesium oxide, hydroxide or carbonate, aluminum or calcium hydroxide, carbonate or silicate; titanium oxide, or composite aluminum/magnesium compounds such as, for instance Al₂O₃.MgO.CO₂(Mg₆Al₂(OH)₁₆CO₃.4H₂O), MgO.Al₂O₃.2SiO₂.nH₂O, or other pharmaceutically acceptable pH-buffering substances such as, for instance the sodium, potassium, calcium, magnesium and aluminum salts of phosphoric, citric or other suitable, weak, inorganic or organic acids.

In one embodiment, the separating layer consists of one or more water soluble inert layers, optionally containing pH-buffering substances. In particular, the separating layer can include, but are not limited to, pharmaceutically acceptable, water soluble, inert compounds or polymers used for film-coating applications such as, for instance sugar, polyethylene glycol, polyvinylpyrrollidone, polyvinyl alcohol, hydroxypropyl cellulose, hydroxymethyl cellulose, hydroxypropyl methylcellulose or the like.

In a particular embodiment, the thickness of the separating layer is not less than 2 μm, for small spherical pellets preferably not less than 4 μm, for tablets, preferably not less than 10 μm.

The separating layer(s) can be applied to the cores—pellets or tablets—by conventional coating procedures in a suitable coating pan or in a fluidized bed apparatus using water and/or conventional organic solvents for the coating solution.

In the case of tablets another method to apply the coating can be performed by the dry-coating technique. First a tablet containing the acid labile compound is compressed as described above. Around this tablet another layer is compressed using a suitable tableting machine. The outer, separating layer, consists of pharmaceutically acceptable, in water soluble or in water rapidly disintegrating tablet excipients. The separating layer has a thickness of not less than 1 mm. Ordinary plasticizers, pigments, titanium dioxide talc and other additives may also be included into the separating layer.

In the case of gelatin capsules the gelatin capsule itself serves as separating layer.

Enteric Coating Layer

The enteric coating layer is applied on to the sub-coated cores by conventional coating techniques such as, for instance, pan coating or fluidized bed coating using solutions of polymers in water and/or suitable organic solvents or by using latex suspensions of said polymers.

In a particular embodiment of the present invention, the enteric coating is in the form of enteric coating on beads or beadlets in a capsule, for example enteric coating on microbeads or microbeadlets in a capsule. Such enteric coatings are described in, for example, U.S. Pat. No. 5,597,564, which is herein incorporated by reference.

Examples of technologies that can be used include the following. JP patent publication No. 05-32543 discloses enteric coated capsules each consisting of a body and a cap containing a drug, said body and cap comprising a particulate matter such as alginic acid dispersed in an agar containing base material. U.S. Pat. No. 4,661,162 discloses an enteric soluble composition comprising an enteric-soluble polymer, such as carboxymethylethyl-cellulose, in admixture with a polyanionic polymer, such as algaric acid, which is soluble in or permeable to liquids having a pH value less than or equal to 2. Certain formulations in the prior art have used a separate layer of a coating agent to coat a pellet core. These coated pellets are thereafter further coated with an additional layer of enteric coating. This technique of providing a separate or second additional coating, i.e., a dual layer, as described in U.S. Pat. No. 4,786,505. The coating processes with multilayer films or tempering processes can be used in the present and are described in, for example, U.S. Pat. Nos. 5,645,858; 5,580,578; 5,681,585; and 5,472,712, and in K. Bauer, “Coated Pharmaceutical Dosage Forms”, Medpharm Scientific Publishers, Stuttgart 1998, B. Sutter, Thesis, University of Dusseldorf, 1987, or in F. N. Christensen, Proceed. Intern. Symp. Contr. Rel. Bioact. Mater. 17, 124, 1990.

See also the following U.S. patents that describe methods to prepare enteric coated tablets: U.S. Pat. No. 6,627,223 entitled “Timed pulsatile drug delivery systems”; U.S. Pat. No. 6,627,219 entitled “Oily capsule preparation and the method for preparing same”; U.S. Pat. No. 6,602,522 entitled “Pharmaceutical formulation for acid-labile compounds”; U.S. Pat. No. 6,586,012 entitled “Taste masked pharmaceutical liquid formulations”; U.S. Pat. No. 6,565,877 entitled “Taste masked compositions”; U.S. Pat. No. 6,555,127 entitled “Multi-spike release formulation for oral drug delivery”; U.S. Pat. No. 6,506,407 entitled “Colon-specific drug release system”; U.S. Pat. No. 6,482,823 entitled “Taste masked pharmaceutical liquid formulations”; U.S. Pat. No. 6,455,052 entitled “Enteric coating, comprising alginic acid, for an oral preparation”; U.S. Pat. No. 6,368,629 entitled “Colon-specific drug release system”; U.S. Pat. No. 6,328,994 entitled “Orally disintegrable tablets”; U.S. Pat. No. 6,326,360 entitled “Bubbling enteric coated preparations”; U.S. Pat. No. 6,312,728 entitled “Sustained release pharmaceutical preparation”; U.S. Pat. No. 5,980,951 entitled “Oral coated active drugs”; U.S. Pat. No. 5,972,389 entitled “Gastric-retentive, oral drug dosage forms for the controlled-release of sparingly soluble drugs and insoluble matter”; U.S. Pat. No. 5,968,554 entitled “Sustained release pharmaceutical preparation”; U.S. Pat. No. 5,882,715 entitled “Method of preparing an oral preparation provided on the outer side with an enteric coating, as well as an oral preparation obtained by the method”; U.S. Pat. No. 5,866,619 entitled “Colonic drug delivery system”; U.S. Pat. No. 5,849,327 entitled “Delivery of drugs to the lower gastrointestinal tract”; U.S. Pat. No. 5,824,339 entitled “Effervescent composition and its production”; U.S. Pat. No. 5,681,584 entitled “Controlled release drug delivery device”; U.S. Pat. No. 5,540,945 entitled “Pharmaceutical preparations for oral administration that are adapted to release the drug at appropriate sites in the intestines”; U.S. Pat. No. 5,525,634 entitled “Colonic drug delivery system”; U.S. Pat. No. 5,525,354 entitled “Pharmaceutical preparation and process for its manufacture”; U.S. Pat. No. 5,468,503 entitled “Oral pharmaceutical preparation released at infragastrointestinal tract”; U.S. Pat. No. 5,430,021 entitled “Hydrophobic drug delivery systems”; U.S. Pat. No. 5,175,003 entitled “Dual mechanism controlled release system for drug dosage forms”; U.S. Pat. No. 4,976,949 entitled “Controlled release dosage form”; U.S. Pat. No. 4,849,227 entitled “Pharmaceutical compositions”; U.S. Pat. No. 4,601,896 entitled “Pharmaceutical capsule compositions and structures for gastric sensitive materials”; U.S. Pat. No. 4,522,625 entitled “Drug dispenser comprising wall formed of semipermeable member and enteric member”; U.S. Pat. No. 4,457,907 entitled “Composition and method for protecting a therapeutic drug”; and U.S. Pat. No. 4,432,966 entitled “Compressed tablets for disintegration in the colon comprising an active ingredient containing nucleus coated with a first layer containing microcrystalline cellulose which is coated with an enteric organic polymer coating”.

The enteric coatings can be made, for example, from one or more layers of fatty acids, such as stearic acid and palmitic acid, wax, shellac, phthalates such as cellulose acetate phthalate, hydroxypropyl methylcellulose phthalate, or polyvinyl acetate phthalate, an acrylic resin which is available in commerce, for example under the trade mark Eudragit™ and acrylic-based Acryl-Eze, and/or mixtures thereof. Further examples include acrylates or copolymers of acrylate and acrylic acid, such as copolymers of methacrylic acid and ethyl acrylate and/or methyl acrylate, for example Eudragit L30D, Eudragit FS 30 D, Eudragit L30D-55, or Eudragit L100-55 of Rohm & Hass or Instacoat EN-Sol, Instacoat EN-HPMC-P, Instacoat EN Super, or Instacoat EN II. In another embodiment, the enteric coating layer contains phtalates, such as poly(vinyl acetate) phthalate-based Sureteric or a latex of cellulose acetophtalate (CAP), such as Aquateric of FMC.

The enteric layer can optionally further comprise one or more pharmaceutically acceptable anti-foaming agents such as a type of silicone, for example simethicone.

The enteric layer can optionally further comprise one or more pharmaceutically acceptable dispersants such as talc, colorants and pigments.

The core, separating layer, or enteric layer can optionally further comprise one or more pharmaceutically acceptable plasticizers, such as triethylcitrate (Citroflex-2), tributylcitrate (Citroflex-4), acetyltributylcitrate (Citroflex-A4), dibutyl sebacate (DBS), diethylphtalate (DEP), acetylated monoglyceride (Myvacet 9-40), polyethylenoglycols or 1,2-propylene glycol. The amount of plasticizer is usually optimized for each enteric coating polymer(s) and is usually in the range of 1-20% of the enteric coating polymer(s).

The core, separating layer, or enteric layer can optionally further comprise one or more pharmaceutically acceptable lubricants such as talc, stearic acid, stearate, such as magnesium stearate, sodium stearyl fumarate, glyceryl behenate, kaolin, aerosol, or colloidal silicon dioxide.

The core, separating layer, or enteric layer can optionally further comprise one or more pharmaceutically acceptable excipients, such as a lactose, starches, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin and castor oil.

The core, separating layer, or enteric layer can optionally further comprise one or more pharmaceutically acceptable adhesives such as polyvinyl pyrrolidone (PVP), povidone, crospovidone, gelatin, hydroxyalkyl cellulose, hydroxymethyl cellulose, hydroxyethyl cellulose (HEC), hydroxypropyl cellulose (HPC), hydroxypropyl methyl cellulose (HPMC), Prosolv, croscarmellose, cross-linked carboxyalkyl cellulose, cross-linked carboxymethyl cellulose, vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit.RTM., copolymer of methyl acrylic acid/methyl methacrylate with polyvinyl acetate phthalate (PVAP).

The core, separating layer, or enteric layer can optionally further comprise one or more pharmaceutically acceptable diluents such as lactose, starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin and castor oil.

Final Dosage Form

The final dosage form is either an enteric coated tablet or capsule or in the case of enteric coated pellets, pellets dispensed in hard gelatin capsules or sachets or pellets formulated into tablets. In one embodiment, for the long term stability during storage that the water content of the final dosage form containing β-D-D4FC (enteric coated tablets, capsules or pellets) is kept low, preferably not exceeding 1.5% by weight.

In one embodiment, the enterically coated composition of the present invention is formulated as disclosed in the examples. In another embodiment, the enterically coated composition of the present invention is formulated as disclosed one or more of the following patents: U.S. Pat. No. 5,464,633, U.S. Pat. No. 5,549,913, U.S. Pat. No. 5,626,874, U.S. Pat. No. 5,891,474, U.S. Pat. No. 6,190,692, U.S. Pat. No. 4,853,230, U.S. Pat. No. 5,585,115, U.S. Pat. No. 6,521,261; U.S. Pat. No. 6,471,994; U.S. Pat. No. 6,358,533; U.S. Pat. No. 6,217,909; U.S. Pat. No. 6,106,865; U.S. Pat. No. 6,103,219; U.S. Pat. No. 5,948,438; U.S. Pat. No. 5,866,166; U.S. Pat. No. 5,858,412; U.S. Pat. No. 5,741,524; U.S. Pat. No. 5,725,884; U.S. Pat. No. 5,725,883, U.S. Pat. No. 5,741,524, U.S. Pat. No. 6,149,942, U.S. Pat. No. 5,800,836, U.S. Pat. No. 5,780,057, U.S. Pat. No. 6,506,407, U.S. Pat. No. 5,500,161, U.S. Pat. No. 5,464,631, or U.S. Pat. No. 6,346,269.

In particular, one embodiment of the invention is directed to enteric compositions of β-D-D4FC formulated as disclosed in U.S. Pat. No. 5,464,633, which teaches pharmaceutical tablets for oral administration suitable to release the active substance consisting essentially of:
a core containing the active substance to be released in the gastric or intestinal tract, a polymeric substance which swells and/or gels and/or erodes on contact with water or aqueous liquids and is selected from the group consisting of hydroxypropylmethylcellulose having a methoxyl content of 22.1% and a viscosity of 15,000 centipoises, crosslinked polyvinylpyrrolidone, crosslinked sodium carboxymethylcellulose, potassium methacrylate-divinyl-benzene copolymer, polyvinylalcohols and beta cyclodextrin and adjuvants and excipients;
a layer applied externally to said core by a compression process said layer being suitable to allow the release of the active substance contained in the core after a definite period of time and being selected from the group consisting of hydroxypropylmethylcellulose having a methoxyl content of 22.1% and a viscosity of 4,000 centipoises, carboxy vinyl polymers, glucans, mannans, xanthans and carboxymethylcellulose and adjuvants and excipients;
   wherein said layer is applied externally to said core and has a thickness of 0.2-4.5 mm which allows the release of said active substance in said core after contact with water or an aqueous liquid for a period of 2 to 3 hours.

Another embodiment of the invention is directed to enteric compositions of β-D-D4FC formulated as disclosed in U.S. Pat. No. 5,549,913, which teaches tablets for controlled release of a drug to be administered orally and for release of said drug at a constant rate with zero order kinetic, said tablet comprising two external layers containing 5-70% by weight of the total weight of said tablet of hydrophilic swelling polymers separated by an interposed layer containing a water soluble polymer in the amount of up to 20% by weight of the total weight of said tablet, said drug being mixed with at least one of said external layers containing said hydrophilic swelling polymers, said interposed layer controlling the release of said drug.

Another embodiment of the invention is directed to enteric compositions of β-D-D4FC formulated as disclosed in U.S. Pat. No. 5,626,874, which teaches controlled release pharmaceutical tablets having a lenticular form consisting of the 3 following over-imposed layers:
a central layer or core (a) comprising an active principle,
2 external barrier layers (b) and (c) respectively upon and under said core (a) limiting the active principle release, each barrier (b) and (c) comprising a gellable and/or erodible polymeric material,
   wherein said barrier layers (b) and (c) have the same or different composition and leave exposed only the lateral surface of the core (a), said exposed lateral surface ranging from 5 to 35% of the total tablet surface.

Another embodiment of the invention is directed to enteric compositions of β-D-D4FC formulated as disclosed in U.S. Pat. No. 5,891,474 and/or U.S. Pat. No. 6,190,692, which teaches delayed time-specific delivery of a pharmaceutically active agent to a patient comprising administering to said patient, a pharmaceutical formulation comprising (a) a core comprising said pharmaceutically active agent, and (b) a swellable polymeric coating layer substantially surrounding said core, that delays the release of said pharmaceutically active agent from said core for a predetermined period of time of about 4 to about 9 hours dependent upon the thickness of said swellable polymeric coating layer; and wherein said swellable polymeric coating layer is provided by alternately (i) wetting said core with a binder solution, and (ii) coating said core with powdered polymeric particles a sufficient number of times to produce a time-specific dosage formulation having the desired thickness of swellable polymeric coating layer.

Another embodiment of the invention is directed to enteric compositions of β-D-D4FC formulated as disclosed in U.S. Pat. No. 4,853,230 to Aktiebolaget Hassle, which teaches pharmaceutical preparations comprising:
(a) an alkaline reacting core comprising an acid-labile pharmaceutically active substance and an alkaline reacting compound different from said active substance, an alkaline salt of an acid labile pharmaceutically active substance, or an alkaline salt of an acid labile pharmaceutically active substance and an alkaline reacting compound different from said active substance;
(b) an inert subcoating which rapidly dissolves or disintegrates in water disposed on said core region, said subcoating comprising one or more layers comprising materials selected from the group consisting of tablet excipients, film-forming compounds and alkaline compounds; and
(c) an enteric coating layer surrounding said subcoating layer, wherein the subcoating layer isolates the alkaline reacting core from the enteric coating layer such that the stability of the preparation is enhanced.

Another embodiment of the invention is directed to enteric compositions of β-D-D4FC formulated as disclosed in U.S. Pat. No. 5,585,115 to Edward H. Mendell Co., Inc., which teaches compositions comprising a particulate agglomerate of microcrystalline cellulose co-processed with silicon dioxide. See also U.S. Pat. Nos. 6,521,261; 6,471,994; 6,358,533; 6,217,909; 6,106,865; 6,103,219; 5,948,438; 5,866,166; 5,858,412; 5,741,524; 5,725,884; and 5,725,883.

Another embodiment of the invention is directed to enteric compositions of β-D-D4FC formulated as disclosed in U.S. Pat. No. 5,741,524, which teaches sustained-release formulations comprising 1) an active agent; 2) an augmented microcrystalline cellulose comprising agglomerated particles of microcrystalline cellulose and a compressibility augmenting agent which
(i) physically restricts the proximity of the interface between adjacent cellulose surfaces,
(ii) inhibits interactions between adjacent cellulose surfaces; or
(iii) accomplishes both (i) and (ii) above; and
3) a matrix comprising a sustained-release carrier to promote sustained-release of said active agent.

Another embodiment of the invention is directed to enteric compositions of β-D-D4FC formulated as disclosed in U.S. Pat. No. 6,149,942 to Melpha A G, which teaches pharmaceutical pellet formulations with a core containing omeprazole in the form of its free base as the active ingredient, optionally with adjuncts, such as binders, sedimentation retarders, e.g. silicon dioxide, and pH correctors, and an enteric coating, wherein the enteric coating optionally contain TiO₂.

Another embodiment of the invention is directed to enteric compositions of β-D-D4FC formulated as disclosed in U.S. Pat. No. 5,800,836, which teaches pelletized sustained release pharmaceutical compositions comprising (a) a core element comprising an active ingredient of low aqueous solubility, a binding agent; and a core seed; and (b) a core coating comprising an enteric polymer; an insoluble polymer; and optionally a plasticizer, such that the active ingredient is released in a controlled fashion over an extended period in the intestine but substantially no release occurs in the acid environment of the stomach and blood levels of active ingredient are maintained within the therapeutic range over an extended period of time.

Another embodiment of the invention is directed to enteric compositions of β-D-D4FC formulated as disclosed in U.S. Pat. No. 5,780,057, which teaches pharmaceutical tablets wherein the active ingredients are released at a controlled rate selectively in the first portion of the gastrointestinal tract, said tablet having a multi-layer structure and comprising:
a) a first layer, which considerably and rapidly swells in the presence of biological aqueous fluids, said swelling resulting in an increase by at least 50% of the total volume of the tablet when coming into contact with the gastric juice, said layer being formed by a compressed granular mixture of biocompatible hydrophilic polymers and at least one highly swellable (superdisintegrating) polymer selected from the group consisting of cross-linked polyvinylpyrrolidone, hydroxypropylcellulose and hydroxypropyl methylcellulose having molecular weight up to 150,000, cross-linked sodium carboxymethylcellulose, carboxymethyl starch, sodium carboxymethyl starch, potassium methacrylate-divinylbenzene copolymer, polyvinyl alcohols, amylose, cross-linked amylose, starch derivatives, microcrystalline cellulose and cellulose derivatives, alpha-, beta- and gamma-cyclodextrin and dextrin derivatives in general, said substances amounting to 1% to 90% of the layer weight
b) a second layer, adjacent to the first and containing the active ingredient, made out of biodegradable and biocompatible polymeric materials and other adjuvants whereby the formulation can be formed by compression and the active ingredient may be released within a time interval that may be predetermined by preliminary tests in vitro;
c) an optional third layer, formed by compression and applied to the second layer, comprising erodible and/or gellable and/or swellable hydrophilic polymers and, being initially impermeable to the active ingredient, acting as a barrier modulating the release of the active ingredient contained in the adjacent 2nd layer, said third layer optionally being identical with the first layer in composition and functional characteristics.

Another embodiment of the invention is directed to enteric compositions of β-D-D4FC formulated as disclosed in U.S. Pat. No. 6,506,407, which teaches oral drug delivery systems for releasing a drug specifically in the colon of the gastrointestinal tract, wherein said system comprises a drug (b) coated with a pharmaceutically acceptable acrylic or cellulosic organic acid-soluble polymer material which dissolves at a pH lower than 6 (a), in an amount of from 2.5% to 40% and a saccharide (c), which rapidly generates an organic acid by the action of enterobacteria in the lower part of the gastrointestinal tract in an amount of from 10% to 99.9%, wherein said composition comprising the drug (b) coated with the organic acid-soluble polymer material (a) and saccharide (c), is further coated with a pharmaceutically acceptable enteric coating polymer material which dissolves at a pH not lower than 6 (d) and wherein said composition when orally administered, is delivered to the lower part of the gastrointestinal tract without releasing the drug (b) at the upper part of the gastrointestinal tract and, at the lower part of the gastrointestinal tract, the polymer (a) coating the drug (b) is dissolved by organic acids generated by degradation of the saccharide (c), by the enterobacteria.

Another embodiment of the invention is directed to enteric compositions of β-D-D4FC formulated as disclosed in U.S. Pat. No. 5,500,161, which teaches microparticles which comprises (i) dispersing a hydrophobic polymer in an aqueous solution in which a substance to be delivered is dissolved, dispersed or suspended; and then (ii) coagulating the polymer together with the substance by impact forces.

Another embodiment of the invention is directed to enteric compositions of β-D-D4FC formulated as disclosed in U.S. Pat. No. 5,464,631, which teaches tamper resistant and tamper evident medicament dosage forms comprising an active pharmaceutical ingredient, pharmaceutically acceptable carrier materials and excipients that are compressed into a generally ovoid, cylindrically shaped medicament caplet that is partially encapsulated by a gelatin capsule, wherein said caplet is of a first color and said gelatin capsule is of a second, different color and wherein said caplet is adhesively bonded or press-fitted into the gelatin capsule.

In one embodiment of the present invention, a unit dosage of the active material is administered once a day. The oral pharmaceutical formulation can be designed to maintain an extended release of the pharmaceutical substance of a minimum of 2 and a maximum of 12 hours, preferably is maintained for a minimum of 4 and a maximum of 8 hours, for example a minimum of 2, 4, or 6 and maximum 6, 8, 10 or 12 hours. Such an extended release preparation may comprise up to 200 mg of the substance, preferably the doses comprise about 25 mg, 50 mg, 75 mg, 100 mg, 150 mg, 200 mg, or 250 mg of the substance.

Combination and/or Alternation Therapy

In a preferred but not necessary embodiment, compounds of the present invention are administered in combination and/or alternation with one or more other anti-HIV agent. In one embodiment the effect of administration of the two or more agents in combination and/or alternation is synergistic.

The emergence of resistant HIV strains has led to the use of combination therapy, the use of two or more drugs as the same time in a drug “cocktail,” which has been shown to be effective at least initially to combat the high risk of resistance to any individual drug. Combination therapy is now the standard of care for people with HIV. It is sometimes called HAART (Highly Active Anti-Retroviral Therapy). Drug resistance most typically occurs by mutation of a gene that encodes for an enzyme used in the viral replication cycle, and most typically in the case of HIV, in either the reverse transcriptase or protease genes. It has been demonstrated that the efficacy of a drug against HIV infection can be prolonged, augmented, or restored by administering the compound in combination and/or alternation with a second, third, fourth, etc., antiviral compound that induces a different mutation(s) from that selected for by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Typically, combination and/or therapy include three selections, including some combination of NRTIs alone or in combination with a PI. The choice of which drugs to combine takes into account synergistic effects of the drug combinations, as well as other sorts of drug-drug interactions that might render a combination less effective or even dangerous.

The additional antiviral agent for the treatment of HIV, in one embodiment, can be a protease inhibitor, a reverse transcriptase inhibitor (a “RTI”), which can be either a synthetic nucleoside reverse transcriptase inhibitor (a “NRTI”) or a non-nucleoside reverse transcriptase inhibitor (a “NNRTI”), and HIV-integrase inhibitor, a fusion inhibitor, or a chemokine inhibitor. In other embodiments, the second (or third) compound can be a pyrophosphate analog, or a fusion binding inhibitor. A list compiling resistance data collected in vitro and in vivo for a number of antiviral compounds is found in Schinazi et al., Mutations in retroviral genes associated with drug resistance, International Antiviral News, Volume 5 (8), International Medical Press 1997.

Potential antiviral agents that can be used in combination and/or alternation with β-D-D4FC of this invention can be screened for their ability to inhibit the relevant HIV enzymatic activity in vitro according to any screening methods known in the art. One can readily determine the spectrum of activity by evaluating the compound in the assays described herein or with another confirmatory assay.

In one embodiment the efficacy of the anti-HIV compound is measured according to the concentration of compound necessary to reduce the plaque number of the virus in vitro, according to methods set forth more particularly herein, by 50% (i.e. the compound's EC₅₀). In preferred embodiments the compound exhibits an EC₅₀ of less than 15 or 10 micromolar.

In preferred embodiments, the compound is administered in combination or alternation with Emtriva (FTC, 2′,3′-dideoxy-3′-thia-5-fluorocytidine); 141W94 (amprenavir, GlaxoWellcome, Inc.); Viramune (nevirapine), Rescriptor (delavirdine); DMP-266 (efavirenz), DDI (2′,3′-dideoxyinosine); 3TC (3′-thia-2′,3′-dideoxycytidine); or DDC (2′,3′-dideoxycytidine). In another preferred embodiment, the phenylindole is administered in combination or alternation with abacavir (1592U89), which is (1S,4R)-4-[2-amino-6-cyclopropyl-amino)-9H-purin-9-yl]-2-cyclopentene-1-methanol succinate, D4T or AZT.

NRTIs, the class that includes the first drug approved by the FDA, that can be used in the present invention, are analogs of normal nucleotides that act as building blocks by reverse transcriptase in the process of assembling DNA from viral RNA. These aberrant nucleoside triphosphates are incorporated into the transcribed DNA chain, preventing elongation, stopping viral replication, or simply act as enzyme inhibitors. NRTIs include AZT (Zidovudine, Retrovir, GlaxoSmithKline), Epivir (3TC, β-L-2′,3′-dideoxy-3′-thiacytidine, GlaxoSmithKline); Emtriva (FTC, β-L-2′,3′-dideoxy-3′-thia-5-fluorocytidine, Gilead Sciences, Inc.); Didanosine (ddI, 2′,3′-dideoxyinosine, Bristol Myers Squibb); Abacavir (Ziagen, GlaxoSmithKline); Stavudine (D4T, 2,3-dideoxy-β-D-glycero-pent-2-eno-furanosyl thymine, Bristol Myers Squibb), and Amdoxovir (DAPD, 2′,3′-dideoxy-3′-oxa-2,6-diaminopurine, Gilead Sciences, Inc).

NNRTIs that can be used in the present invention include Sustiva (efavirenz; Bristol Myers Squibb) and nevirapine (Viramune, BI-587, Boeringer Ingelheim). The NNRTIs can produce moderate levels of intolerance related to rash and central nervous system (CNS) effects, as well as infrequent but serious toxicity, (including severe cutaneous reactions and liver toxicity) and a low genetic barrier to resistance. NNTRIs are nevertheless prescribed because they can be tolerated for long periods if initial serious reactions do not occur. Development of NNRTIs continues as a source of therapeutic alternatives to HIV positive individuals who have developed resistance to those NNRTIs already on the market.

Another class of drugs that target the viral protease, an enzyme responsible for processing HIV-fusion polypeptide precursors, can be used in the present invention. In HIV and several other retroviruses, the proteolytic maturation of the gag and gag/pol fusion polypeptides (a process indispensable for generation of infective viral particles) has been shown to be mediated by a protease that is, itself, encoded by the pol region of the viral genome. Y. Yoshinaka, et al., Proc. Natl. Acad. Sci. USA, 82:1618-1622 (1985); Y. Yoshinaka, et al., J. Virol., 55:870-873 (1985); Y. Yoshinaka, et al., J. Virol., 57:826-832 (1986); and K. von der Helm, Proc. Natl. Acad. Sci., USA, 74:911-915 (1977). Inhibition of the protease has been shown to inhibit the processing of the HIV p55 in mammalian cell and HIV replication in T lymphocytes. T. J. McQuade, et al., Science, 247:454 (1990). FDA approved protease inhibitors include Saquinavir (Invirase® Fortovase®, Roche); Ritonavir (Norvir®, Abbott); Indinavir (Crixivan®, Merck); Nelfinavir (Viracept®, Agouron); Amprenavir (Agenerase®, GlaxoSmithKline); Lopinavir (Kaletra®, Abbott); Atazanavir® (BMS 232632, Bristol-Myers Squibb). Other protease inhibitors in human trials include: GW433908 (GlaxoSmithKline), Tipranavir® (Boehringer Ingelheim); and TMC114 (Tibotec Virco).

Preferred protease inhibitors include indinavir ({1(1,S,2R),5(S)]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythro-pentoamide sulfate; Merck), nelfinavir (Agouron), ritonavir (Abbott), saquinavir (Roche) and DMP-450 {[4R-4(r-a,5-a,6-b,7-6)]-hexahydro-5,6-bis(hydroxy)-1,3-bis(3-amino)phenyl]methyl)-4,7-bis(phenylmethyl)-2H-1,3-diazepin-2-one}-bismesylate (Triangle Pharmaceuticals, Inc.).

Another class of drugs that can be used in the present invention, are compounds that affect the integration of viral DNA into the host chromosome. Considerable effort has been directed to develop drugs targeting integrase, the viral protein responsible for integration. Integrase is an attractive target for antivirals because, unlike protease and reverse transcriptase, there are no known counterparts in the host cell. Goldgut, Y, et al., Biochemistry, 96(23)13040-13043 (1999). Two drugs in early clinical trials including S-1360 (Shionogi Pharmaceuticals and GlaxoSmithKline) and L-870810 (Merck).

Fusion or entry inhibitors also can be used in the present invention. Unlike NNRTIs, NRTIs and PIs, which are only active against HIV after it has entered a cell, fusion inhibitors prevent the virus from entering the cell. HIV entry can be broken down into three basic steps: (1) HIV binding via the gp120 envelope protein to the CD4 molecule on the Th1 cell surface; (2) a change in envelope protein conformation that leads to gp120 binding to a second receptor (either CCR5 or CXCR4); and 3) gp41-mediated fusion of the viral envelope with the cell membrane, completing viral entry. At least one inhibitor targeting each step in this pathway is currently in clinical development. Only one entry inhibitor, Fuzeon® (Trimeris), has been approved by the FDA for treatment of HIV. Fuzeon is a peptide therapeutic which works by binding to gp41. T1249 (Trimeris), another fusion inhibitor that works by binding to gp41, is also in human trials. Fusion inhibitors that bind to T-cell proteins are also in development, including PRO-542 (Progenics Pharmaceuticals) and BMS 806 (Bristol Myers Squibb). AMD070 (AnorMed) targets the CXCR4 chemokine receptor and is currently in Phase I. Development of another AnorMed fusion inhibitor, AMD3100, was halted in May of 2001 as a result of poor clinical results. SCH C (Schering Plough) is a CCR5 antagonist that blocks the interaction between the V3 loop of gp120 and the CCR5 receptor. The FDA has allowed further clinical development of this agent despite adverse cardiac events in early clinical trials.

Antisense drugs, another new class of therapeutics that can be used in the present invention, block expression of viral genes using antisense oligonucleotides. Viruses are particularly suitable targets for antisense therapy because they carry genetic information distinct from the host cells. At present, two antisense drugs are in early clinical trials, including HGTV43 (Enzo Biochem) and GEM-92 (Hybridon). Antisense technology, however, has produced only one marketable drug in the twenty years since it was first developed. Several HIV antisense programs have been abandoned as a result of drug delivery issues and dosage concerns. Whereas each of the aforementioned therapies for HIV launch an offensive attack against the virus, attention has also been given to defensive strategies for enhancing the immune system of HIV infected patients. It is known that patients mount a characteristic immune response with weeks to months of infection with HIV, including production of HIV-specific antibodies and expansion of HIV-1 specific CD4 and CD8 T cells. Moog, C et al., J Virol, 71(5):3734-41 (1997); Robert-Guroff, et al., Nature, 316(6023):72-4 (1985).

Strategies for enhancing the immune response that can be used in the present invention are varied. One strategy involves cytokine manipulation. Cytokines are the chemical messengers of the immune system, and include small proteins and biological factors such as interleukins, chemokines, lymphokines, interferons and other signaling molecules such as tumor necrosis factor. While the role of cytokines in disease progress is not clearly understood, cytokine profiles are clearly disturbed. Levels of certain cytokines (e.g., IL-1, IL-6, TNP-alpha, inteferons-alpha and gamma) increase, while others (e.g., IL-2) decrease. The best-known cytokine therapeutic is interleukin-2 (IL-2, Aldesleukin, Proleukin, Chiron Corporation). IL-2 is often used in combination with antiretroviral drugs or and during therapeutic “breaks” from antiretroviral therapy. Multikine (Cel-Sci Corporation) is a mixture of several different cytokines. HE2000 (Hollis-Eden Pharmaceuticals) is in Phase I/II trials. Reticulose (Advanced Viral Research Corporation) is a nucleic acid that stimulates the cell-killing arm of the immune system.

A second strategy for enhancing the immune system involves passive immunization using blood products derived from other animals or humans infected with HIV. Passive immunization has a long history in the treatment of disease beginning with the use of serum therapy in the late 1800's. Behring and Kitasato were the first to use passive immunization in the treatment of diphtheria in 1890. At that time, serum was not known to contain antibodies; rather it was only observed that serum produced a beneficial therapeutic effect. In the 1920's and 30's, sera produced in animals (e.g., horse, rabbit, sheep) was used to treat patients with measles and scarlet fever. Serum therapy declined in popularity with the discovery of antibodies, and the development of strategies such as pooling and monoclonal antibody technology. Passive immunization against HIV has involved treating patients with heterologous neutralizing antibodies (HNabs). The term “heterologous” refers to the source of neutralizing antibodies as “other,” and can refer to other humans or animals. In general, HNabs are prepared by purification from the serum of HIV positive individuals, or by exposing a particular animal to HIV, permitting Nabs to develop, and then isolating those Nabs from the animal serum.

HNabs produced from the sera of HIV positive individuals have been used to treat HIV. Karpas, A et al., Proc Natl Acad Sci, 85:9234-7 (1988); Levy, J et al. Blood, 84(7):2130-5 (1994); Vittecoq, D et al., Proc Natl Acad Sci, 92(4):1195-9 (1995). U.S. Pat. No. 4,863,730 (Karpas) teaches a method for treating from HIV positive individuals in which plasma is obtained from HIV positive patients and then processed to provide a preparation having a high titer of HNabs. Karpas distinguishes the unprocessed plasma (plasma as it is derived by separation from an individual's blood, for example by centrifugation) from the “processed plasma” composition of the invention (which is processed to remove substantially all non-fluidic components other than certain antibodies). While some clinical reports suggest therapeutic benefit (i.e, reduced HIV viremia and delay in onset of disease), this method is not considered broadly applicable and manufacturing is not scalable.

It has also been suggested to prompt HIV positive patients to produce neutralizing antibodies against HIV by exposing them to viruses similar to, but less pathogenetic than, HIV. U.S. Pat. No. 6,033,672 (Douvas) teaches the use of caprine arthritis-encephalitis virus (CAEV), a lentivirus found in goats with low pathogenicity in humans, for prophylactic or therapeutic purposes against HIV. CAEV capable of infecting humans have been found in people of Mexican descent, and CAEV positive individuals which develop anti-CAEV antibodies have been shown to react to both the CAEV gp135 surface glycoprotein and the HIV gap120 envelope glycoprotein to substantially neutralize the virus. Although not heterologous in nature, these neutralizing antibodies are intended to supplement the immune response.

HNabs have also been produced in animals. WO 97/02830 (Davis) teaches methods and compositions for treating HIV involving administration of neutralizing antibodies produced in goats. The goats are immunized with viral lysates. The blood of the immunized animal is then collected, and processed by standard extraction and purification methods (e.g., ammonium sulfate precipitation followed by dialysis or gel filtration) to produce an immunomodulatory composition enriched for HNabs. Davis distinguishes between the straight, untreated serum used to monitor goat antibody production in vivo from the serum composition used to treat the patient, which is a polyclonal immunoglobulin concentrate. Other filings by Davis include WO 01/60156, WO 02/07760 and U.S. 2002/006022. All teach processing of immunized animal sera to obtain serum extracts suitable for in vivo use. The Davis method and composition have been used to treat patients outside of the United States, as widely reported in such media sources as the Washington Post (Apr. 9, 2000) and on Dateline Houston (Sep. 18, 1998). This method, however, has not been shown to have the ability to lower viral load over the long term. Furthermore, the viral lysate is a single clone of laboratory virus that is known to be more susceptible than the HIV found in infected individuals, and therefore the composition has not been formulated to treat the condition as it exists in humans.

Generation of HNabs in animals permits rational design of immunogens. The HIV-1 envelope glycoprotein gp120 mediates receptor binding and is a major target for neutralizing antibodies. Purified gp120, however, has been shown to elicit type specific neutralizing antibodies, making it unsuitable for production of broadly neutralizing HNabs. Immunogen design therefore turned toward other epitopes. U.S. Pat. No. 6,456,172 (Gelder et al.) teaches methods and compositions for treating HIV involving administration of HNabs which recognize viral epitopes that fail to elicit neutralizing antibodies in humans when encountered through natural infection. The neutralizing antibodies are produced in goats, and antisera are processed to produce the therapeutic compositions. More specifically, the antisera is fractionated with octanoic acid, centrifuged and then filtered. The immunoglobulin fraction is then purified over a series of columns, filtered and then brought to the desired concentration of neutralizing antibody. The neutralizing antibody composition of Gelder et al. corresponds to HRG214, a polyclonal antibody preparation manufactured by Vironyx Corporation, recently the subject of a Phase I clinical trial. Dezube, B J et al., J Infect Dis, 187(3):500-3 (2003).

Nonlimiting examples of compounds that can be administered in combination or alternation with the compounds of the present invention to augment the properties of the drug on administration include abacavir: (1S,4R)-4-[2-amino-6-cyclopropyl-amino)-9H-purin-9-yl]-2-cyclopentene-1-methanol succinate (1592U89, a carbovir analog; GlaxoWellcome); BILA 1906: N-{1 S-[[[3-[2S-{(1,1-dimethylethyl)amino]carbonyl}-4R-]3-pyridinylmethyl)thio]-1-piperidinyl]-2R-hydroxy-1S-(phenylmethyl)propyl]-amino]carbonyl]-2-methylpropyl}-2-quinolinecarboxamide (Bio Mega/Boehringer-Ingelheim); BILA 2185: N-(1,1-dimethylethyl)-1-[2S-[[2-2,6-dimethyl-phenoxy)-1-oxoethyl]amino]-2R-hydroxy-4-phenylbutyl]4R-pyridinylthio)-2-piperidine-carboxamide (Bio Mega/Boehringer-Ingelheim); BM+51.0836:triazoloiso-indolinone derivative; BMS 186,318: aminodiol derivative HIV-1 protease inhibitor (Bristol-Myers-Squibb); d4API: 9-[2,5-dihydro-5-(phosphonomethoxy)-2-furanyl]-adenine (Gilead); stavudine: d4T, 2′,3′-dide-hydro-3′-deoxythymidine (Bristol-Myers-Squibb); HBY097: S-4-isopropoxycarbonyl-6-methoxy-3-(methylthio-methyl)-3,4-dihydroquinoxalin-2-(1H)-thione; HEPT: 1-[(2-hydroxy-ethoxy)methyl]6-(phenylthio)-thymine; KNI-272: (2S,3S)-3-amino-2-hydroxy-4-phenylbutyric acid-containing tripeptide; L-697,593; 5-ethyl-6-methyl-3-(2-phthalimido-ethyl)pyridin-2(1H)-one; L-735,524: hydroxyamino-pentane amide HIV-1 protease inhibitor (Merck); L-697,661: 3-{[(−4,7-dichloro-1,3-benzoxazol-2-yl)methyl]amino}-5-ethyl-6-methylpyridin-2(1H)-one; L-FDDC: (−)-β-L-5-fluoro-2′,3′-dideoxycytidine; L-FDOC: (−)-β-L-5-fluoro-dioxolane cytosine; nevirapine: 11-cyclopropyl-5,11-dihydro-4-methyl-6H-dipyridol[3,2-b:2′,3′-e]diazepin-6-one (Boehringer-Ingelheim); PFA: phosphonoformate (foscamet; Astra); PMEA: 9-(2-phosphonylmethoxyethyl)adenine (Gilead); PMPA: (R)-9-(2-phosphonylmethoxypropyl)adenine (Gilead); Ro 31-8959: hydroxyethylamine derivative HIV-1 protease inhibitor (Roche); RPI-3121: peptidyl protease inhibitor, 1-[(3s)-3-(n-alpha-benzyloxycarbonyl)-1-asparginyl)-amino-2-hydroxy-4-phenylbutyryl]-n-tert-butyl-1-proline amide; 2720: 6-chloro-3,3-dimethyl-4-(isopropenyloxycarbonyl)-3,4-dihydroquinoxalin-2 (1H)thione; SC-52151: hydroxyethylurea isostere protease inhibitor (Searle); SC-55389A: hydroxyethyl-urea isostere protease inhibitor (Searle); TIBO R82150: (+)-(5S)-4,5,6,7-tetrahydro-5-methyl-6-(3-methyl-2-butenyl)-imidazo-[4,5,1-jk]-[1,4]-benzodiazepin-2(1H)-thione (Janssen); TIBO 82913: (+)-(5S)-4,5,6,7,-tetrahydro-9-chloro-5-methyl-6-(3-methyl-2-butenyl)imidazo[4,5,1,jk]-[1,4]-benzodiazepin-2-(1H)-thione (Janssen); TSAO-m3T:[2′,5′-bis-O-(tert-butyldimethylsilyl)-3′-spiro-5′-(4′-amino-1′,2′-oxathiole-2′,2′-dioxide)]-β-D-pentofaranosyl-N-3-methylthymine; U90152: 1-[3-[(1-methylethyl)-amino]2-pyridinyl]-4-[[5-[(methylsulphonyl)-amino]-1H-indol-2yl]-carbonyl]-piperazine; UC: thiocarboxanilide derivatives (Uniroyal); UC-781=N-[4-chloro-3-(3-methyl-2-butenyloxy)phenyl]-2-methyl-3-furancarbothioamide; UC-82=N-[4-chloro-3-(3-methyl-2-butenyloxy)phenyl]-2-methyl-3-thiophenecarbothioamide; VB 11,328: hydroxyethyl-sulphonamide protease inhibitor (Vertex); VX-478: amprenavir, 141W94, hydroxyethyl-sulphonamide protease inhibitor (Vertex/Glaxo Wellcome); XM 323: cyclic urea protease inhibitor (Dupont Merck), famciclovir, gancyclovir and penciclovir. In another embodiment, the phenylindole is administered in combination with the protease inhibitor LG 1350.

Preferred antiviral agents that can be used in combination and/or alternation with the compounds disclosed herein for HIV therapy include 3TC; FTC, foscamet; carbovir, acyclovir, interferon, stavudine, and β-D-dioxolane nucleosides such as β-D-dioxolanylguanine (DXG), β-D-dioxolanyl-2,6-diaminopurine (DAPD), and β-D-dioxolanyl-6-chloropurine (ACP).

The following drugs have been approved by the FDA or are currently in clinical trials for use in the treatment of HIV infection, and therefore in one embodiment, can be used in combination and/or alternation with the compounds of the present invention.

| Drug Name | Manufacturer |
|---|---|
| 3TC, Epivir ® brand lamivudine | GlaxoSmithKline |
| abacavir generic Ziagen ®, ABC, or 1592U89 | GlaxoSmithKline |
| ABC, Ziagen ® brand abacavir, or 1592U89 | GlaxoSmithKline |
| ABT-378/r, or Kaletra ® brand lopinavir/ritonavir | Abbott Laboratories |
| AG-1549, S-1153, or capravirine (CPV) | Pfizer |
| AG1661, Remune ® brand HIV-1 Immunogen, or Salk vaccine | Immune Response Corp. |
| Agenerase ® brand amprenavir (APV) 141W94, or VX-478 | GlaxoSmithKline |
| aldesleukin generic Proleukin ®, or Interleukin-2 (IL-2) | Chiron Corporation |
| amdoxovir, or DAPD | Gilead Sciences |
| amprenavir generic Agenerase ®, APV, 141W94, or VX-478 | GlaxoSmithKline |
| APV, Agenerase ® brand amprenavir, 141W94, or VX-478 | GlaxoSmithKline |
| atazanavir generic Reyataz ™, or BMS-232632 | Bristol-Myers Squibb |
| AZT, Retrovir ® brand zidovudine (ZDV) | GlaxoSmithKline |
| Bis(POC) PMPA, Viread ® brand tenofovir DF | Gilead Sciences |
| BMS-232632, or Reyataz ™ brand atazanavir | Bristol-Myers Squibb |
| BMS-56190, or DPC-083 | Bristol-Myers Squibb |
| calanolide A | Sarawak Medichem |
| capravirine (CPV), AG-1549, or S-1153 | Pfizer |
| Combivir ® brand zidovudine + lamivudine, or AZT + 3TC | GlaxoSmithKline |
| CPV (capravirine), AG-1549, or S-1153 | Pfizer |
| Crixivan ® brand indinavir (IDV) or MK-639 | Merck & Co. |
| d4T, Zerit ® brand stavudine, or BMY-27857 | Bristol-Myers Squibb |
| DAPD, or amdoxovir | Gilead Sciences |
| ddC, or Hivid ® brand zalcitabine | Hoffmann-La Roche |
| ddI, Videx ® brand didanosine, or BMY-40900 | Bristol-Myers Squibb |
| delavirdine generic Rescriptor ®, DLV, or U-90152S/T | Pfizer |
| didanosine generic Videx ®, ddI, or BMY-40900 | Bristol-Myers Squibb |
| DLV, Rescriptor ® brand delavirdine, or U-90152S/T | Pfizer |
| DPC-083, or BMS-56190 | Bristol-Myers Squibb |
| Droxia ® brand hydroxyurea (HU) | Bristol-Myers Squibb |
| efavirenz generic Sustiva ®, or EFV | Bristol-Myers Squibb |
| EFV, Sustiva ® brand efavirenz | Bristol-Myers Squibb |
| emtricitabine generic Emtriva ™, or FTC | Gilead Sciences |
| Emtriva ® brand emtricitabine, or FTC | Gilead Sciences |
| enfuvirtide generic Fuzeon ™, or T-20 | Trimeris and Hoffmann-La |
| | Roche |
| Epivir ® brand lamivudine, or 3TC | GlaxoSmithKline |
| epoetin alfa (erythropoietin) generic Procrit ® | Ortho Biotech |
| erythropoietin (epoetin alfa) generic Procrit ® | Ortho Biotech |
| Fortovase ® brand saquinavir (Soft Gel Cap), or SQV (SGC) | Hoffmann-La Roche |
| fosamprenavir, or GW-433908, or VX-175 | GlaxoSmithKline |
| FTC, or Emtriva ® brand emtricitabine | Gilead Sciences |
| Fuzeon ™ brand enfuvirtide, or T-20 | Trimeris and Hoffmann-La |
| | Roche |
| GW-433908, or fosamprenavir, or VX-175 | GlaxoSmithKline |
| HE2000, or alpha-epibromide | HollisEden |
| | Pharmaceuticals |
| HIV-1 Immunogen generic Remune ®, Salk vaccine, or AG1661 | Immune Response Corp. |
| Hivid ® brand zalcitabine, or ddC | Hoffmann-La Roche |
| HU, or Droxia ® brand hydroxyurea | Bristol-Myers Squibb |
| hydroxyurea generic Droxia ®, or HU | Bristol-Myers Squibb |
| IDV, Crixivan ® brand indinavir, or MK-639 | Merck & Co. |
| IL-2 (Interleukin-2), or Proleukin ® brand aldesleukin | Chiron Corporation |
| indinavir generic Crixivan ®, IDV, or MK-639 | Merck & Co. |
| Interleukin-2 (IL-2), or Proleukin ® brand aldesleukin | Chiron Corporation |
| Invirase ® brand saquinavir (Hard Gel Cap), SQV (HGC), or | Hoffmann-La Roche |
| Ro-31-8959 | |
| Kaletra ® brand lopinavir/ritonavir, or ABT-378/r | Abbott Laboratories |
| lamivudine generic Epivir ®, or 3TC | GlaxoSmithKline |
| lopinavir/ritonavir generic Kaletra ®, or ABT-378/r | Abbott Laboratories |
| MK-639, Crixivan ® brand indinavir (IDV) | Merck & Co. |
| nelfinavir generic Viracept ®, NFV, or AG-1343 | Pfizer |
| nevirapine generic Viramune ®, NVP, or BI-RG-587 | Boehringer Ingelheim |
| NFV, Viracept ® brand nelfinavir, or AG-1343 | Pfizer |
| Norvir ® brand ritonavir (RTV), or ABT-538 | Abbott Laboratories |
| NVP, Viramune ® brand nevirapine, or BI-RG-587 | Boehringer Ingelheim |
| PNU-140690, or tipranavir | Boehringer Ingelheim |
| PRO-542 | Progenics |
| | Pharmaceuticals |
| Procrit ® brand epoetin alfa (erythropoietin) | Ortho Biotech |
| Proleukin ® brand aldesleukin, or Interleukin-2 (IL-2) | Chiron Corporation |
| Remune ® brand HIV-1 Immunogen, or Salk vaccine | Immune Response Corp. |
| Rescriptor ® brand delavirdine (DLV), or U-90152S/T | Pfizer |
| Retrovir ® brand zidovudine (ZDV), or AZT | GlaxoSmithKline |
| Reyataz ™ brand atazanavir, or BMS-232632 | Bristol-Myers Squibb |
| Salk vaccine, Remune ® brand HIV-1 Immunogen, or AG1661 | Immune Response Corp. |
| saquinavir (Hard Gel Cap) generic Invirase ®, SQV (HGC), or | Hoffmann-La Roche |
| Ro-31-8959 | |
| saquinavir (Soft Gel Cap) generic Fortovase ®, or SQV (SGC) | Hoffmann-La Roche |
| SCH-C | Schering-Plough |
| Serostim ® brand somatropin | Serono Laboratories |
| somatropin generic Serostim ® | Serono Laboratories |
| SQV (HGC), Invirase ® brand saquinavir (Hard Gel Cap), or | Hoffmann-La Roche |
| Ro-31-8959 | |
| SQV (SGC), or Fortovase ® brand saquinavir (Soft Gel Cap) | Hoffmann-La Roche |
| stavudine generic Zerit ®, d4T, or BMY-27857 | Bristol-Myers Squibb |
| Sustiva ® brand efavirenz (EFV) | Bristol-Myers Squibb |
| T-1249 | Trimeris and |
| | Hoffmann-La Roche |
| T-20, or Fuzeon ™ brand enfuvirtide | Trimeris and |
| | Hoffmann-La Roche |
| TDF, tenofovir DF generic Viread ™, or Bis(POC) PMPA | Gilead Sciences |
| tenofovir DF (TDF) generic Viread ®, Bis(POC) PMPA | Gilead Sciences |
| tipranavir, or PNU-140690 | Boehringer Ingelheim |
| TMC-114 | Tibotec-Virco Group |
| TMC-125 | Tibotec-Virco Group |
| Trizivir ® brand abacavir + zidovudine + lamivudine (ABC + | GlaxoSmithKline |
| AZT + 3TC) | |
| Videx ® brand didanosine, ddI, or BMY-40900 | Bristol-Myers Squibb |
| Videx ® EC brand didanosine (ddI): delayed-release capsules | Bristol-Myers Squibb |
| Viracept ® brand nelfinavir (NFV), or AG-1343 | Pfizer |
| Viramune ® brand nevirapine (NVP), or BI-RG-587 | Boehringer Ingelheim |
| Viread ® brand tenofovir DF, or Bis(POC) PMPA | Gilead Sciences |
| VX-175, or fosamprenavir, or GW-433908 | GlaxoSmithKline |
| zalcitabine generic Hivid ®, or ddC | Hoffmann-La Roche |
| ZDV, Retrovir ® brand zidovudine, or AZT | GlaxoSmithKline |
| Zerit ® brand stavudine, d4T, or BMY-27857 | Bristol-Myers Squibb |
| Ziagen ® brand abacavir (ABC), or 1592U89 | GlaxoSmithKline |
| zidovudine generic Retrovir ®, AZT, or ZDV | GlaxoSmithKline |

The disclosed combination and alternation regiments are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV.

The following drugs have been approved by the FDA for use in the treatment of complications of HIV infection and AIDS, which can be used in combination and/or alternation with the compounds of the present invention.

### Drugs Used to Treat Complications of HIV/AIDS

| | | | Manufacturer |
|---|---|---|---|
| Brand Name | Generic Name | Use | Name |
| Abelcet, | Amphotericin B, ABLC | antifungal for | various |
| Ambisome | | aspergillosis | |
| Bactrim, Septra | sulfamethoxazole and | antiprotozoal | various |
| | trimethoprim | antibiotic for | |
| | | Pneumocystis carinii | |
| | | pneumonia treatment | |
| | | and prevention | |
| Biaxin, Klacid | clarithromycin | antibiotic for | Abbott |
| | | Mycobacterium avium | Laboratories |
| | | prevention and | |
| | | treatment | |
| Cytovene | ganciclovir, DHPG | antiviral for CMV | Roche |
| | | retinitis | |
| DaunoXome | daunorubicin- | chemotherapy for | Gilead |
| | liposomal | Kaposi's sarcoma | |
| Diflucan | fluconazole | antifungal for | Pfizer |
| | | candidiasis, | |
| | | cryptococcal | |
| | | meningitis | |
| Doxil | doxorubicin | chemotherapy for | Ortho Biotech |
| | hydrochloride- | Kaposi's sarcoma | |
| | liposomal | | |
| Famvir | famciclovir | antiviral for | Novartis |
| | | herpes | |
| Foscarnet | foscavir | antiviral for | Astra |
| | | herpes, CMV | Pharmaceuticals |
| | | retinitis | |
| Gamimune N | immune globulin, | immune booster to | Bayer |
| | gamma globulin, IGIV | prevent bacterial | Biologicals |
| | | infections in | |
| | | children | |
| Intron A | interferon alfa-2b | Karposi's sarcoma, | Schering |
| | | hepatitis C | |
| Marinol | dronabinol | treat appetite loss | Roxane |
| | | | Laboratories |
| Megace | megestrol acetate | treat appetite, | Bristol Myers- |
| | | weight loss | Squibb |
| Mepron | atovaquone | antiprotozoal | GlaxoSmithKline |
| | | antibiotic for | |
| | | Pneumocystis carinii | |
| | | pneumonia treatment | |
| | | and prevention | |
| Mycobutin, | rifabutin | antimycobacterial | Adria |
| Ansamycin | | antibiotic for | Pharmaceuticals |
| | | Mycobacterium avium | |
| | | prevention | |
| NebuPent | pentamidine | antiprotozoal | Fujisawa |
| | | antibiotic for | |
| | | Pneumocystis carinii | |
| | | pneumonia prevention | |
| Neutrexin | trimetrexate glucuronate | antiprotozoal | MedImmune |
| | and leucovorin | antibiotic for | |
| | | Pneumocystis carinii | |
| | | pneumonia treatment | |
| Panretin gel | alitretinoin gel 0.1% | AIDS-related Karposi's | Ligand |
| | | sarcoma | Pharmaceuticals |
| Procrit, Epogen | erythropoetin, EPO | treat anemia related | Amgen |
| | | to AZT therapy | |
| Roferon A | interferon alfa-2a | Karposi's sarcoma and | Roche |
| | | hepatitis C | |
| Serostim | somatropin rDNA | treat weight loss | Serono |
| Sporanox | itraconazole | antifungal for | Janssen |
| | | blastomycosis, | Pharmaceuticals |
| | | histoplasmosis, | |
| | | aspergillosis, and | |
| | | candidiasis | |
| Taxol | paclitaxel | Karposi's sarcoma | Bristol Myers- |
| | | | Squibb |
| Valcyte | valganciclovir | antiviral for CMV | Roche |
| | | retinitis | |
| Vistide | cidofovir, HPMPC | antiviral for CMV | Gilead |
| | | retinitis | |
| Vitrasert | ganciclovir insert | antiviral for CMV | Bausch & Lomb |
| implant | | retinitis | |
| Vitravene | fomivirsen sodium | antiviral for CMV | Isis |
| intravitreal | injection | retinitis | Pharmaceuticals |
| injectable | | | |
| Zithromax | azithromycin | antibiotic for | Pfizer |
| | | Mycobacterium avium | |

Several products have been allowed to proceed as Investigational New Drugs (IND) by the FDA for the treatment of complications of HIV infection and AIDS. Therefore, the following drugs can be used in combination and/or alternation with the compounds of the present invention.
Trimetrexate glucuronate for the treatment of Pneumocystis carinii pneumonia in AIDS patients who cannot tolerate standard forms of treatment.
Ganciclovir for the treatment of cytomegaloviras retinitis in AIDS patients.
Aerosolized pentamidine for the prevention of Pneumocystis carinii pneumonia in AIDS patients.
Erythropoietin for the treatment of zidovudine-related anemia.
Atovaquone for the treatment of AIDS patients with Pneumocystis carinii pneumonia who are intolerant or unresponsive to trimethoprim-sulfamethoxazole.
Rifabutin for prophylaxis against Mycobacterium avium complex bacteremia in AIDS patients.
Vistide intravenous cidofovir for HIV-infected persons with relapsing cytomegalovirus (CMV) retinitis that has progressed despite treatment (Hoffmann-La Roche).
Serostim, a mammalian derived recombinat human growth hormone, for the treatment of AIDS-related wasting (Serono Laboratories).

In general, during alternation therapy, an effective dosage of each agent is administered serially, whereas in combination therapy, effective dosages of two or more agents are administered together. The dosages will depend on such factors as absorption, biodistribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges for anti-HIV compounds, including nucleoside derivatives (e.g. D4T, DDI, and 3TC) or protease inhibitors, for example, nelfinavir and indinavir, can be found in the scientific literature and in the Physicians Desk Reference. Many examples of suitable dosage ranges for other compounds described herein are also found in public literature or can be identified using known procedures. These dosage ranges can be modified as desired to achieve a desired result.

The following working examples provide a further understanding of the method of the present invention. These examples are of illustrative purpose, and are not meant to limit the scope of the invention.

### EXAMPLES

### Example 1

Enteric Formulation Comprising β-D-D4FC A preferred β-D-D4FC enteric formulation is a tablet formulation comprising a) a core consisting of D-D4FC and a pharmaceutically acceptable excipient; b) an optional separating layer; c) an enteric layer and a pharmaceutically acceptable excipient; d) an optional finishing layer.

The following example demonstrates the preparation of such formulation.

50 mg β-D-D4FC Enteric Coated Tablet

| Composition | | |
|---|---|---|
| β-D-D4FC | 50.00 | mg |
| Sodium Bicarbonate | 44.50 | mg |
| Microcrystalline Cellulose | 96.00 | mg |
| Crospovidone | 8.00 | mg |
| Magnesium Stearate | 1.50 | mg |
| Core Tablet Weight | 200.00 | mg |
| Enteric layer | | |
| Opadry II White, Y-30-18037 | 4.00 | mg |
| Sureteric, YAE-6-18107 | 0.00 | mg |
| Purified Water, USP | | |
| 30% Simethicone Emulsion | | |
| Total | 234.00 | mg |

### Example 2

Enteric Formulation Comprising β-D-D4FC—Wet Granulation

The β-D-D4FC enteric coated tablets, 100 mg (wet-granulation), are prepared by enterically coating a core tablet containing β-D-D4FC and commonly used excipients. B-D-D4FC is dry mixed with silicified microcrystalline cellulose, mannitol, croscarmellose sodium, and hydroxyproply cellulose in a GPCG-5 fluid-bed dryer and then wet-granulated using an aqueous phosphate buffer as the binder solution. The granulation is dried and then milled through a FitzMill Model M5 mill. The granulation is blended with croscarmellose sodium in a 40-L Blender Bohle and then blended with magnesium sterate. Core tablets are compressed on a JCMCO tablet press. In a coating process, using an O'Hara coating pan with 15″ drum, the core tablets are coated with an aqueous opadry white solution until a 3% weight gain is achieved. The coated core tablets are then dried. To form the enteric coating solution, simethicone is mixed with water and then acryl-eze is dissolved. The enteric coating solution is applied to the coated core tablets in the O'Hara coating pan until an 8% weight gain is acheived, and then the enteric-coated tablets are dried. The tablets are packaged in white high-density polyethylene (HDPE), 30-cc, round bottles and a CRC closure with SFG.75 ISTS liner. One tablet is packaged per bottle. The unit formula for β-D-D4FC enteric coated tablets, 100 mg (wet-granulation), is shown in the Table 1 below.

TABLE 1
Unit Formula of the Reverset Enteric Coated
Tablets, 100 mg (wet-granulation)

| | | | |
|---|---|---|---|
| | | Quantity/Unit | Reference to |
| Ingredient | Function | (mg/tablet) | Standards |

Core Tablet

| | | | |
|---|---|---|---|
| Reverset | Active | 100.00 | |
| Silicified | Filler/Binder | 66.25 | NF |
| Microcrystalline | | | |
| Cellulose | | | |
| (ProSolv (S/MCC) | | | |
| 90) | | | |
| Mannitol | Soluble Filler | 50.53 | USP |
| Hydroxypropyl | Binder | 6.25 | NF |
| Cellulose (Klucel) | | | |
| Croscarmellose | Disintegrant | 7.50 | NF |
| Sodium (Ac-Di-Sol) | | | |
| Sodium Phosphate | Buffering Agent | 17.47 | USP |
| Dibasic, | | | |
| Heptahydrate | | | |
| Magnesium Stearate | Lubricant | 2.00 | USP, NF |
| Purified Water^{a} | Solvent | qs | USP |
| Subtotal | | 250.00 | |

Subcoating^{b}

| | | | |
|---|---|---|---|
| Opadry White | Subcoating | 7.50 | DMF No. 721^{d} |
| (YS-1-18177-A) | | | |
| Purified Water^{a} | Solvent | qs | USP |
| Subtotal | | 257.50 | |
| Enteric Coating^{c} | | | |
| Acryl-eze MP | Enteric Coating | 18.73 | DMF No. 721^{d} |
| (Formula 93018429) | | | |
| Simethicone | Antifoam Agent | 1.87 | USP |
| Purified Water^{a} | Solvent | qs | USP |
| Total | | 278.10 | |

^{a}Removed during processing
^{b}3% coating weight gain. Solution prepared in excess.
^{c}8% coating weight gain. Solution prepared in excess.
^{d}A Drug Master File (DMF) Letter of Authorization is provided in Appendix 4.
qs = Quantity sufficient

### Example 3

Enteric Formulation Comprising β-D-D4FC

The following example demonstrates another preparation comprising a) a core consisting of D-D4FC and a pharmaceutically acceptable excipient; b) an optional separating layer; c) an enteric layer and a pharmaceutically acceptable excipient; d) an optional finishing layer.

50 mg β-D-D4FC Enteric Coated Tablet

| Composition | | |
|---|---|---|
| β-D-D4FC | 50.000 | mg |
| Prosolv (SMCC 50) | 33.125 | mg |
| Mannitol | 29.375 | mg |
| Hydroxypropyl Cellulose | 3.125 | mg |
| Croscarmellose Sodium | 2.500 | mg |
| Sodium Diphosphate, Dibasic | 4.625 | mg |
| Magnesium Stearate | 1.000 | mg |
| Core Tablet Weight | 125.000 | mg |
| Separation layer | | |
| Opadry White, YS-1-18177-A | 2.500 | mg |
| Coated Tablet Weight | 127.500 | mg |
| Enteric layer | | |
| Eudragit L30 D-55 | 7.500 | mg |
| Triethyl Citrate | 1.130 | mg |
| Talc | 3.750 | mg |
| 30% Simethicone Emulsion | | |
| Sodium Hydroxide (pellets) | | |
| Purified Water, USP | | |
| Total | 139.880 | mg |

### Example 4

Enteric Formulation Comprising β-D-D4FC

The following example demonstrates another preparation comprising a) a core consisting of D-D4FC and a pharmaceutically acceptable excipient; b) an optional separating layer; c) an enteric layer and a pharmaceutically acceptable excipient; d) an optional finishing layer.

| Composition | |
|---|---|
| β-D-D4FC | 27.68% |
| Sodium Phosphate, Dibasic Anhydrous | 3.19% |
| PVP K29/32 | 8.96% |
| 20/25 mesh Sugar Cores | 29.37% |
| Separation layer | |
| HPMC E-5 | 2.77% |
| Simethicone | 0.35% |
| Enteric layer | |
| Acryl-EZE MP | 27.68% |
| TOTAL | 100.00% |

Finishing Layer
Size 0 white opaque capsule shells (Cardinal Health)

### Example 5

Enteric Formulation Comprising β-D-D4FC—Coated Pellets

β-D-D4FC enteric-coated pellets are prepared using a layering suspension of β-D-D4FC. β-D-D4FC is dispersed in aqueous phosphate buffer. The suspension is milled through a DYNO-MILL. Povidone is added to the milled material and mixed. In a fluid-bed layering process, sugar spheres of mesh size 20-25 are heated to 40-50° C., the suspension is then applied, and the layered pellets are dried. A subcoating of hydroxypropyl methylcellulose is applied as an aqueous solution to the layered pellets in a fluid-bed layering process and dried. Simethicone is added to water, mixed, and Acryl-eze MP is added and dispersed during the mixing. The Acryl-eze MP suspension is applied to the subcoated pellets in a fluid-bed layering process, and the enteric-coated pellets are dried. Two-piece (size 0), hard-shell gelatin capsules are filled with the coated pellets to a target fill weight calculated based on an in-process assay test to achieve a 100.0-mg dose of β-D-D4FC per capsule. The capsules are packaged in white high-density polyethylene (HDPE) 30-cc round bottles and a CRC Closure with SFG.75 I PRT liner. One capsule is packaged per bottle. The unit formula for 100-mg β-D-D4FC capsules is shown in Table 2.

TABLE 2
Composition of 100 mg Reverset Enteric-Coated Capsules Product

| | | Quantity/Unit | Reference to |
|---|---|---|---|
| Ingredient | Function | (mg/capsule) | Standards |
| Layering | | | |
| Reverset | Active | 100.0 | |
| Sodium Phosphate | Buffering | 6.10 | USP, Ph. Eur. |
| Dibasic, Heptahydrate^{a} | Agent | (11.52) | |
| Povidone (Plasdone | Binder | 32.26 | USP, Ph. Eur. |
| K-29/32) | | | |
| 20/25 Sugar Spheres | Base Pellets | 106.11 | NF, Ph. Eur. |
| Purified Water^{b} | Solvent | (431.68) | USP |
| | | Subtotal 244.47 | |
| Sub-Coating^{c} | | | |
| Hydroxypropyl | Sub-Coating | 10.00 | NF, Ph. Eur. |
| Methylcellulose | | | |
| (Methocel E5) | | | |
| Purified Water^{b} | Solvent | (123.29) | USP |
| | | Subtotal 254.47 | |
| Enteric Coating^{d} | | | |
| Acryl-eze MP | Enteric Coating | 102.68 | DMF No. 721^{c} |
| (Formula 93018429) | | | |
| Simethicone | Antifoam | 1.27 | USP, Ph. Eur. |
| | Agent | | |
| Purified Water^{b} | Solvent | (415.82) | USP |
| | | Total 358.42 | |
| Encapsulation | | | |
| Empty Gelatin | | 1 capsule shell | USP, Ph. Eur. |
| Capsules^{f} | | | |

^{a}Associated water removed during processing, (includes water weight)
^{b}Removed during processing
^{c}4% coating weight gain. Solution prepared in excess.
^{d}40% coating weight gain. Solution prepared in excess.
^{e}A DMF letter of authorization is provided in Appendix 4.
^{f}Capsule manufactured by Pharmaphil Inc.

### Example 6

Pharmacokinetic Profile of β-D-D4FC

Pooled, PHA-stimulated PBMCs were used for the measurement of intracellular phosphorylation of β-D-D4FC. Cells were extracted in methanol, centrifuged, and the supernatant filtered. HPLC was accomplished using a WAX column with an acetonitrile/ammonium acetate buffer elution system to separate mono, di and tri-phosphorylated versions of β-D-D4FC. Mass spectrometric analyses were performed on a Sciex API 4000 triple quadruple mass spectrometer, using the fluorine-substituted cytosine fragment for quantitation.

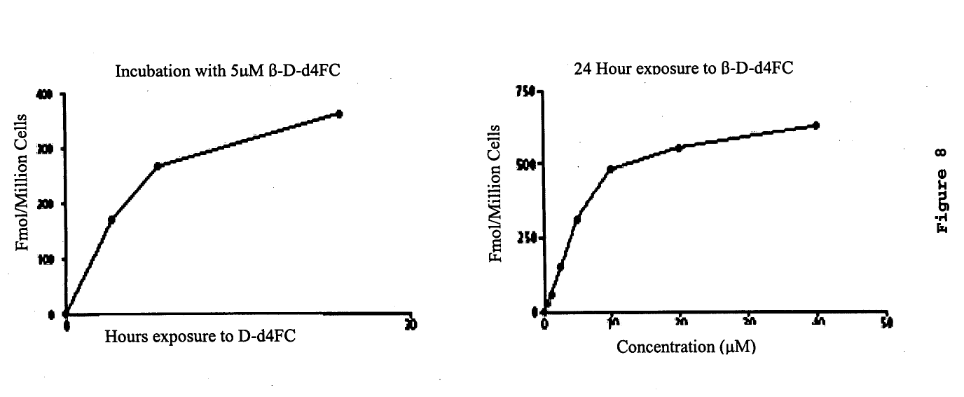

Intracellular conversion to β-D-D4FC-TP plateaus at extracellular concentrations of β-D-D4FC-TP of ˜10 μM. Uptake is rapid and accumulates to ˜10% of parent β-D-D4FC-TP in 8-10 hours (see FIG. 8).

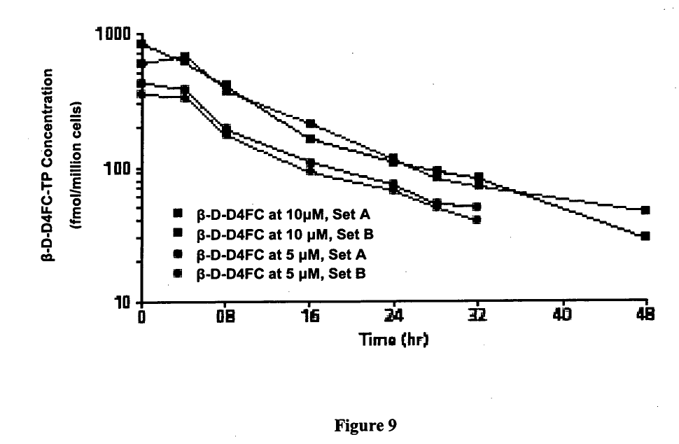

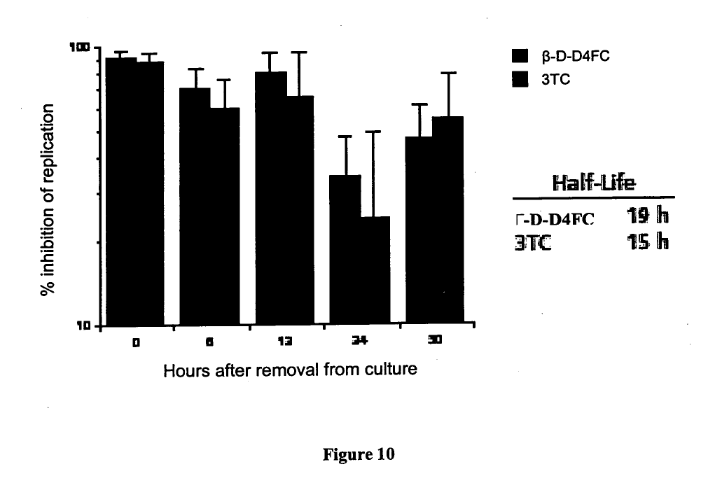

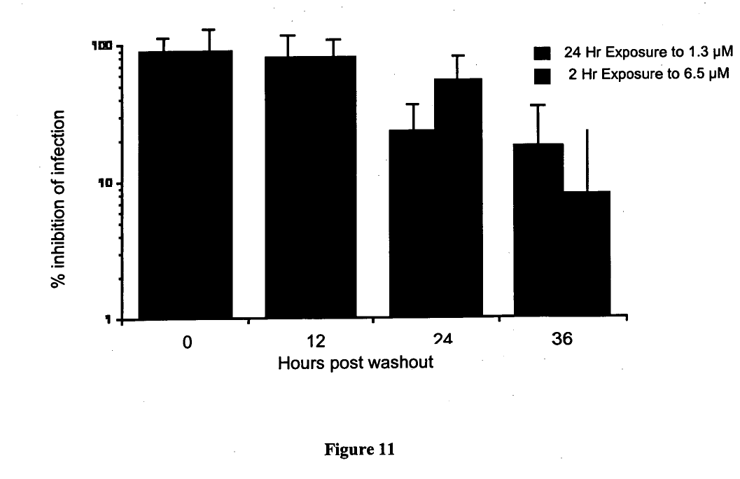

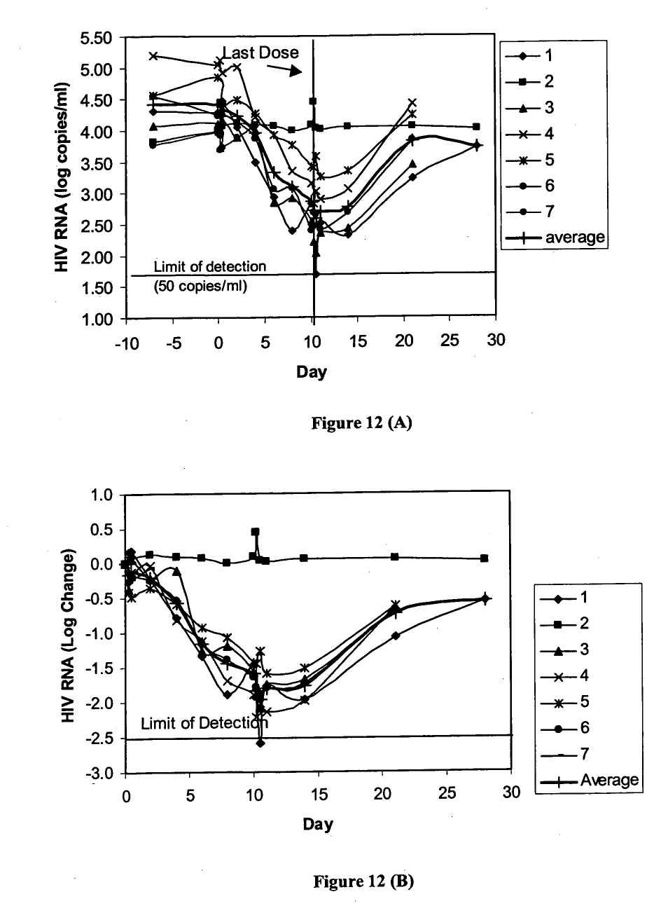

PBMCs were incubated with 5 or 10 micromolar β-D-D4FC, and incubated for 24 hours. Timed samples were analyzed for remaining β-D-D4FC-TP content to determine the intracellular half-life (see FIG. 9). The functional half-life was determined using MT-2 cells incubated 24 hours with 1.3 μM D-d4FC or 3TC (˜IC₉₀ level for wild type). See FIGS. 10 (24 hour exposure to D-d4FC or 3TC) and 11 (comparison of 2 versus 24 hours exposures to D-d4FC).

β-D-D4FC has rapid uptake and conversion to the active metabolite (β-D-D4FC-triphosphate), which has an intracellular half-life of 13 to 17 hours. Taken together, the data suggests a transient level of ˜2.5 μM β-D-D4FC will be sufficient for inhibition of wild type for many hours. Pharmacokinetics in the rhesus monkey suggests low clearance of parent β-D-D4FC, and a plasma half-life longer than either AZT or 3TC.

### Example 7

Antiviral Activity and Cytotoxicity of β-D-D4FC

β-D-2′,3′-didehydro-2′,3′-dideoxy-5-fluorocytidine (β-D-D4FC, DPC 817, RVT, Reverset) is a cytidine nucleoside analog that is active against wild type (WT) HIV-RT. Further, as shown above, β-D-D4FC has a long intracellular half-life and a target plasma level of only 5 micromolar of compound is necessary to achieve >90% suppression of relevant viruses.

An assay was performed to determine the ability of β-D-D4FC-triphosphate to terminate polymerization catalyzed by purified HIV-1 reverse transcriptase (RT). See Table 3. The ability of β-D-D4FC to inhibit wild type virus replication was assessed in MT-2 cells or PBMCs via p24 antigen detection, yield reduction, or measurement of HIV-RT activity. The concentration causing 90% suppression of replication was designated the IC₉₀. Where measured, IC₅₀ values were 5-fold lower. The target concentration is that concentration where most mutant variants tested are suppressed by 90% or more. The concentration of 5 μM is the target plasma concentration as most, mutant variants tested were suppressed ≧90% in vitro.

TABLE 3
D-d4FC (DPC 817) is a Potent and
Selective Inhibitor of HIV-1 RT

| | | | ddC-TP | | | |
|---|---|---|---|---|---|---|
| Polymerase | β-D-D4FC-TP | | IC₅₀ (μM) | | 3TC-TP | |
| HIV RT | 0.14 | 0.007 | ND | | 0.57 | 0.05 |
| DNA Pol β | 1.20 | 0.08 | 0.25 | 0.05 | 10.3 | 1.68 |
| DNA Pol γ | 0.74 | 0.07 | 0.07 | 0.03 | 43.1 | 6.53 |

Triphosphates of nucleosides were tested against pure polymerases using tritiated dCTP incorporation.

β-D-D4FC-triphosphate inhibits wild type purified RT with an IC₅₀ value of 67±40 nM. β-D-D4FC inhibits wild type laboratory and clinical isolates of HIV-1 with an average IC₉₀ value of 855±400 nM.

Inhibition of DNA Pol γ was not associated with mitochondrial toxicity in mouse primary bone marrow cells at concentrations <1 mM. See Table 4.

TABLE 4
The Cytotoxicity of β-D-D4FC is Low

| Cell line | TC₅₀ μM |
|---|---|
| 293 cells (24 h) | >30 |
| Huh7 cells (48 h) | >30 |
| BHK-21 cells(30 h) | >30 |
| MT-2 cells (72 h) | >220 |
| Vero cells (96 h) | >100 |
| PBMCs (144 h) | >100 |

Cytotoxicity was measured by reduction of the formazan dye MTT, which is catalyzed by mitochondrial succinate dehydrogenase.

HepG2 cells were incubated for 14 days with the active agent. and mitDNA and rRNA levels were determined by quantitative real-time PCR. The results are tabulated in

TABLE 5
Mitochondrial Toxicity Testing of β-D-D4FC
and Related Cytidine Analogs

| | Concentration | % Change in | % Change in | % Change in |
|---|---|---|---|---|
| Compound | (μM) | mitDNA | rRNA | Lactic Acid* |
| β-D-D4FC | 1 | 0 | 0 | +12 |
| | 10 | −1.6 | 0 | −2 |
| L-D4FC | 1 | −15 | 0 | +25 |
| | 10 | −74.2 | −75.5 | +147 |
| ddC | 1 | −99.7 | −65.4 | +390 |
| | 10 | −99.7 | −73.6 | +492 |
| 3TC | 1 | 0 | −0.3 | +23 |
| | 10 | −9 | −0 | +5 |
| No Drug | | 0 ± 20 | 0 ± 0 | 0 ± 25 |

*Lactic acid change is normalized for cytotoxicity.

Single doses of β-D-D4FC were well tolerated at all doses tested and no serious adverse events were observed at any dose level. All adverse events were mild in nature—fatigue and headache were the most commonly reported adverse events. All adverse events occurred no more frequently than with placebo. There was no relationship between dose and the number or intensity of the adverse events.

Median effective concentration and combination index (C.I.) values for Racivir (RCV), β-D-D4FC (Reverset, RVT), D4T, DDI alone and in combination with Racivir in acutely HIV-1 infected human PBM cells (Day 5) were calculated. The experiment was conducted in duplicate in T25 flasks.

The combination of Racivir and Reverset at a ratio of 1:1 was additive at all levels. However, Racivir and Reverset at a ratio of 1:5 appeared slightly antagonistic at low levels and at 90 and 95% inhibition, the interaction was additive. The combination of Racivir and D4T at a ratio of 1:1 and 1:3 were synergistic at all levels. The combination of Racivir and DDI at a ratio of 1:20 was antagonistic at 50% inhibition, additive and synergistic at all higher levels. See Table 6.

TABLE 6
Median effective concentration and combination index (C.I.) values for Racivir (RCV), β-D-D4FC (Reverset, RVT), D4T, DDI alone
and in combination with Racivir in acutely HIV-1 infected human PBM cells (Day 5). Assay 3.14.03 (1), 3.27.03 (2), 4.03.03 (3)

| | Parameter^{a} | | | | C.I. at Fₐ^{b} of | | | |
|---|---|---|---|---|---|---|---|---|
| Treatment (drug ratio) | m ± SE | EC₅₀ (μM) | EC₉₀ (μM) | R | 0.50 | 0.75 | 0.90 | 0.95 |
| Racivir (1) | 0.87 ± 0.14 | 0.014 | 0.17 | 0.97 | | | | |
| Racivir (2) | 1.4 ± 0.14 | 0.047 | 0.22 | 0.99 | | | | |
| Racivir (3) | 0.88 ± 0.10 | 0.017 | 0.21 | 0.98 | | | | |
| β-D-D4FC (1) | 1.1 ± 0.12 | 0.079 | 0.63 | 0.98 | | | | |
| β-D-D4FC (2) | 1.3 ± 0.20 | 0.24 | 1.3 | 0.97 | | | | |
| D4T (3) | 1.2 ± 0.08 | 0.05 | 0.35 | 0.99 | | | | |
| DDI (3) | 1.2 ± 0.02 | 0.38 | 2.2 | 0.99 | | | | |
| Racivir + β-D-D4FC | 0.85 ± 0.13 | 0.26 | 0.97 | 0.92 ± 0.21 | 1.0 ± 0.21 | 1.1 ± 0.19 | 1.2 ± 0.17 | |
| 1:1 (1) | 0.019 | | | | 0.83 ± 0.16 | 0.89 ± 0.17 | 0.95 ± 0.16 | 1.0 ± 0.15 |
| Racivir + β-D-D4FC | 1.3 ± 0.19 | 0.16 | 0.88 | 0.97 | 1.4 ± 0.25 | 1.5 ± 0.30 | 1.5 ± 0.38 | 1.6 ± 0.48 |
| 1:5 (2) | | | | | 1.1 ± 0.22 | 1.1 ± 0.26 | 1.2 ± 0.32 | 1.2 ± 0.38 |
| Racivir + D4T (3) | 1.1 ± 0.14 | 0.11 | 0.98 | 0.62 ± 0.46 | 0.53 ± 0.54 | 0.47 ± 0.67 | 0.43 ± 0.80 | |
| 1:1 | 0.015 | | | | 0.56 ± 0.44 | 0.48 ± 0.51 | 0.43 ± 0.64 | 0.40 ± 0.81 |
| Racivir + D4T (3) | 1.2 ± 0.08 | 0.12 | 0.99 | 0.59 ± 0.14 | 0.50 ± 0.08 | 0.43 ± 0.08 | 0.40 ± 0.08 | |
| 1:3 | 0.019 | | | | 0.53 ± 0.13 | 0.45 ± 0.08 | 0.40 ± 0.07 | 0.37 ± 0.07 |
| Racivir + DDI (3) | 1.8 ± 0.19 | 0.84 | 0.99 | 1.7 ± 0.16 | 0.98 ± 0.16 | 0.61 ± 0.22 | 0.46 ± 0.29 | |
| 1:20 | 0.24 | | | | 1.3 ± 0.12 | 0.82 ± 0.13 | 0.55 ± 0.19 | 0.42 ± 0.25 |

^{a}m is the slope ± S.E., EC₅₀ is the median effective concentration, and R is the correlation coefficient, as determined from the median effect plot.
^{b}C.I. < 1, equal to 1 or > 1 indicates synergy, additivity and antagonism respectively. Fₐ is a component of the median effect equation referring to the fraction of the system affected (e.g., 0.50 means the C.I. at a 50% reduction of RT activity). C.I. values were determined for a mutually non-exclusive interaction.
(values in italics are for mutually exclusive interaction, which is less rigorous).

### Example 8

Antiviral Activity of Reverset Against AZT and 3TC-resistant Variants

Nucleosides comprise the backbone of HAART regimens. Because of overuse and misuse, resistance to many nucleosides is present in many experienced, and some therapy-naive patients. AZT and 3TC resistance, typified by changes in reverse transcriptase residues 41, 67, 70, 184, 215, 219 represent clinically relevant viral targets for new nucleoside analogs. Therefore, nucleoside analog reverse transcriptase inhibitors (NRTIs) with improved activity against clinically relevant resistant viruses, i.e., “second generation” nucleoside analogs, are needed to construct effective regimens for ARV-experienced individuals. Some criteria for a “second generation” nucleoside analog are as follows: improved potency towards mutant variants that are clinically relevant in nucleoside-experienced patients; plasma pharmacokinetics with QD or BID dosing to achieve levels consistent with 90% inhibition of replication; and uptake efficiency and intracellular half life providing intracellular tri-phosphate levels in excess of the Kᵢ for WT and mutant RTs.

β-D-2′,3′-didehydro-2′,3′-dideoxy-5-fluorocytidine (β-D-D4FC, DPC 817, RVT, Reverset) is a cytidine nucleoside analog that is active against wild type (WT) HIV-RT and retains activity against many mutant variants, including 3TC and AZT-resistant HIV-1 variants.

| | | |
|---|---|---|
| Compound | Kᵢ WT | Kᵢ M184V |

| | | | | |
|---|---|---|---|---|
| β-D-D4FC-TP | 0.1 | μM | 0.3 | μM |
| 3TC-TP | 0.95 | μM | 85 | μM |
| Kₘ for dC-TP | 7 | μM | 9 | μM |

β-D-D4FC was tested against panels of site-directed recombinant viruses encoding NRTI resistance mutations and recombinant viruses containing the RT gene derived from clinical samples of individuals on nucleoside analog therapy. The ability of β-D-D4FC to inhibit wild type virus replication as well as HIV replication of site-directed mutant recombinant viruses was assessed in MT-2 cells or PBMCs via p24 antigen detection, yield reduction, or measurement of HIV-RT activity. The ability of β-D-D4FC to inhibit recombinant viruses containing RT and Protease sequences from non Clade B isolates were assayed using a reporter cell line (Hertogs and Larder, Virco Nev.).

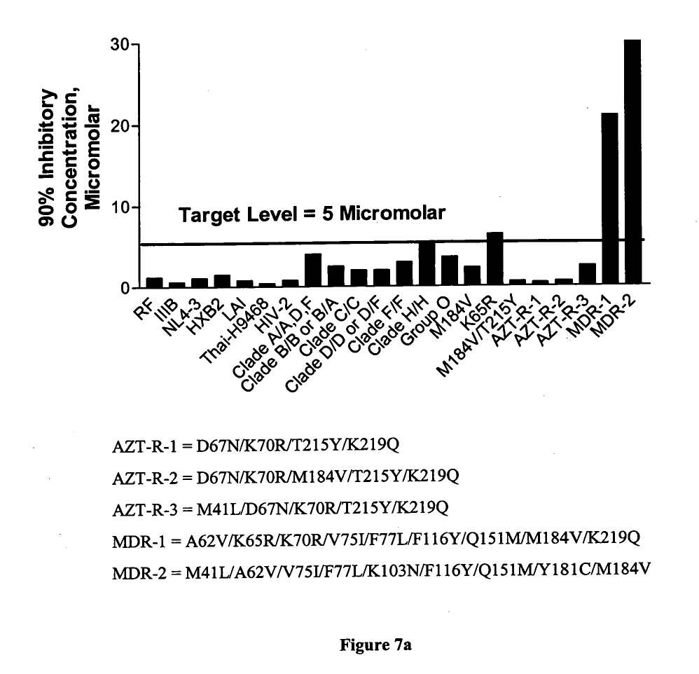

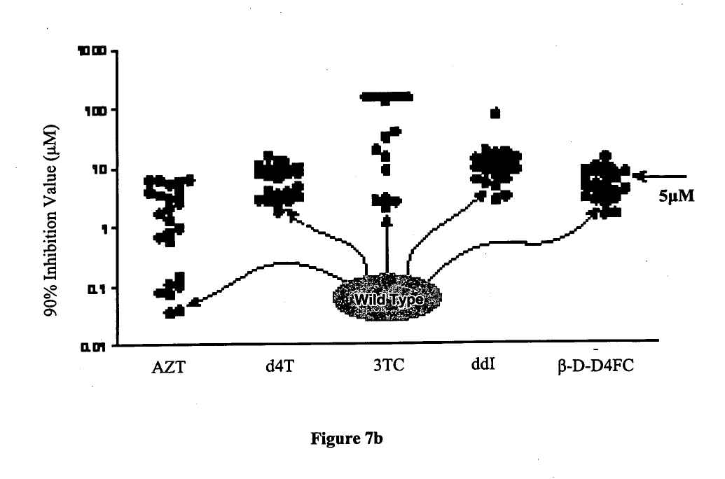

A panel of 22 viruses was constructed in the HXB2 background using the Protease and RT sequences from clinical isolates to determine Virco Profiling against recombinant clinical isolates (see FIGS. 7a and 7b). Viruses contained 2 to 17 mutations in RT frequently associated with nucleoside resistance, including: M184V, M41L, D67N, T215Y. IC₅₀ values were determined using a high through-put reporter virus assay. IC₉₀ values were calculated by multiplying IC₅₀ values by 5.0.

β-D-D4FC showed less than 5-fold reduction in activity against recombinant viruses containing as many as 10 mutations, and including M41L, M184V, D67N, L74V, K70R, T215Y or K219Q. β-D-D4FC was only weakly active against multi-drug resistant strains containing Q151M or D69S insertions plus at least 5 additional mutations. Therefore, β-D-D4FC combines this favorable resistance profile with rapid uptake and conversion to the active metabolite (β-D-D4FC-triphosphate), which has an intracellular half-life of 13 to 17 hours.

The Kᵢ value for β-D-D4FC-TP for wild type HIV-RT was found to be 0.1 μM., while the Kᵢ value for MI84V mutant RT was found to be 0.3 μM. See Table 7.

TABLE 7
β-D-D4FC: Comparisons to Other Nucleosides

| Parameter | β-D-D4FC | | ddI | | d4T | | 3TC | |
|---|---|---|---|---|---|---|---|---|
| Average IC₉₀ | 5.5 | μM | 12.6 | μM | 6.8 | μM | >93 | μM |
| for AZT/3TC-R | | | | | | | | |
| virus | | | | | | | | |
| Plasma Cₘₐₓ | 5 | μM (target) | 4.2 | μM | 2.7 | μM | 6.6 | μM |
| Plasma t_{1/2} | 6-15 | h | 1.5 | h | 1.7 | h | 5-7 | h |
| Intracellular | 0.5 | μM | 0.12 | μM | 1.0 | μM | 17 | μM |
| [Nuc-PPP] in | | | | | | | | |
| vitro | | | | | | | | |
| Intracellular | 13-17 | h | 25-40 | h | 3.5 | h | 8-16 | h |
| half life | | | | | | | | |
| Intracellular | 0.5 | μM (target) | <0.5 | μM | 0.05 | μM | 6-10 | μM |
| [Nuc-PPP] in | | | | | | | | |
| patients | | | | | | | | |
| Kᵢ for Wild | 0.1 | μM | 0.08-8 | μM | 0.05-0.3 | μM | 1 | μM |
| type RT | | | | | | | | |

| | | | | | | | |
|---|---|---|---|---|---|---|---|
| Kᵢ for mutant | 0.3-0.5 | μM | ND | 3 | μM | 85 | μM |
| RT | | | | | | | |

Plasma PK for ddI, d4T and 3TC are inadequate
Intracellular kinetics for ddI, d4T and 3TC are inadequate.

Therefore, β-D-D4FC may be useful as a component of HAART regimens in individuals with resistance to older NRTI agents. β-D-D4FC can combat viral resistance through inhibiting more than 80% of the clinically significant HIV mutant strains, including AZT and 3TC resistant variants. Antiviral profiling established a target value of 5 micromolar parent compound for >90% suppression of AZT, 3TC and co-resistant variants. Much higher concentrations are required for significant inhibition of MDR strains.

### Example 9

Single Dose 8-D-D4FC—24 Hour Study

β-D-D4FC was administered as a single oral dose to HIV-1 infected males at doses of 10, 25 or 50 mg as buffered solutions or 50, 100 or 200 mg as enteric-coated tablets. In a double-blind, placebo-controlled, randomized, cross-over study-evaluated escalating single oral doses of β-D-D4FC (10 mg-200 mg) in HIV-1 infected males subjects to characterize the safety, tolerability, and pharmacokinetics of single oral doses of β-D-D4FC. Total of 18 HIV-1 infected males, aged 18-55 years, enrolled, 6 per treatment cohort. Each subject had a CD4-lymphocyte count ≧50 cells/mm³ and was treatment free (i.e. off NRTIs, NNRTIs, and/or PIs for at least 4 weeks prior to study drug administration).

TABLE 8
Subject Demographics

| Category | |
|---|---|
| Planned/entered/discontinued | 18/18/0 |
| Mean Age in Years | 39.1 |
| (Range) | (24-51) |
| Male/Female | 18/0 |
| Race | |
| Caucasian | 16 |
| Black | 2 |

In Cohort 1, subjects received either 10 mg or 25 mg of β-D-D4FC or placebo as a buffered solution. In Cohort 2, subjects received either placebo or 50 mg of β-D-D4FC as coated tablets, or as a buffered solution, to determine the bioavailability of coated tablets versus buffered solution. In Cohort 3, subjects received either 100 mg, or 200 mg, or placebo.

| | |
|---|---|
| Dosing | |
| Period | Dose |

COHORT 1

| | |
|---|---|
| 1 | 10 mg (N = 4) or placebo (N = 2) |
| 2 | 10 mg (N = 2), 25 mg (N = 2) or |
| | placebo (N = 2) |
| 3 | 25 mg (N = 4) or placebo (N = 2) |

COHORT 2

| | |
|---|---|
| 1 | 50 mg BSF* (N = 4) or placebo (N = 2) |
| 2 | 50 mg BSF (N = 2), 50 mg EC**(N = 2) or |
| | placebo (N = 2) |
| 3 | 50 mg EC (N = 4) or placebo (N = 2) |

COHORT 3

| | |
|---|---|
| 1 | 100 mg (N = 4) or placebo (N = 2) |
| 2 | 100 mg (N = 2), 200 mg (N = 2) or |
| | placebo (N = 2) |
| 3 | 200 mg (N = 4) or placebo (N = 2) |

*BSF = buffered solution formulation,
**EC = enteric coated tablet

Blood samples were drawn prior to dosing and over a 24 hour period following drug administration and the plasma concentrations of β-D-D4FC and its metabolite, 5-fluorocytosine (5-FC) and 5-fluorouracil (5-FU), were determined using LC/MS/MS detection methods. The data was analyzed using non-compartmental and two-compartmental models. See Tables 9-10.

TABLE 9
Pharmacokinetic Results for β-D-D4FC

| | | Parameter | | | | |
|---|---|---|---|---|---|---|
| Dose | | Cₘₐₓ | Tₘₐₓ | AUC | C_{12 h} | T ½ |
| (mg) | | (μM) | (h) | (μM*h) | (μM) | (h) |
| 10 | sol. | 0.87 ± 0.13 | 1.0 ± 0.0 | 3.57 ± 0.55 | 0.06 ± 0.02 | 6.8 ± 4.2 |
| 25 | sol. | 1.76 ± 0.48 | 1.4 ± 1.1 | 8.12 ± 1.47 | 0.13 ± 0.05 | 15.6 ± 12.4 |
| 50 | sol. | 5.2 ± 1.1 | 1.0 ± 0.3 | 28.03 ± 4.51 | 0.48 ± 0.12 | 13.8 ± 2.4 |
| 50 | tab. | 4.4 ± 1.6 | 2.0 ± 0.8 | 25.88 ± 8.3 | 0.49 ± 0.19 | 14.3 ± 3.1 |
| 100 | tab. | 4.95 ± 1.2 | 2.9 ± 1.0 | 32.71 ± 6.85 | 0.62 ± 0.21 | 11.3 ± 1.6 |
| 200 | tab. | 7.7 ± 1.6 | 3.3 ± 1.6 | 52.5 ± 9.2 | 1.13 ± 0.36 | 12.0 ± 0.6 |

Data analyzed using a non-compartmental model

TABLE 10
Pharmacokinetic Results for 5FC

| | | Parameter | | | | |
|---|---|---|---|---|---|---|
| Dose | | Cₘₐₓ | Tₘₐₓ | AUC | C_{12 h} | T ½ |
| (mg) | | (μM) | (h) | (μM*h) | (μM) | (h) |
| 10 | sol. | 0.06 ± 0.03 | 1.0 ± 0.3 | 0.39 ± 0.26 | 0.02 ± 0.00 | 6.4 ± 4.2 |
| 25 | sol. | 0.21 ± 0.19 | 1.8 ± 0.9 | 1.52 ± 0.87 | 0.04 ± 0.02 | 9.5 ± 5.2 |
| 50 | sol. | 0.64 ± 0.33 | 1.3 ± 0.3 | 4.57 ± 2.76 | 0.48 ± 0.12 | 5.8 ± 1.5 |
| 50 | tab. | 0.23 ± 0.32 | 2.4 ± 0.9 | 1.98 ± 3.28 | 0.03 ± 0.02 | 8.6 ± 4.8 |
| 100 | tab. | 0.20 ± 0.11 | 3.7 ± 0.8 | 1.93 ± 0.68 | 0.07 ± 0.02 | 18.6 ± 10.1 |
| 200 | tab. | 2.46 ± 3.17 | 3.3 ± 3.2 | 14.43 ± 13.12 | 0.33 ± 0.24 | 11.2 ± 7.1 |

5FC, the primary metabolite of β-D-D4FC, was determined in plasma using LC/MS/MS methodology. No 5FU was detected at any dose.

TABLE 11
Projected and Actual Human PK Values

| | Cₘₐₓ (μM) | | AUC (μM · h) | |
|---|---|---|---|---|
| Dose (mg) | Projected | Actual | Projected | Actual |
| 10 | 0.63 | 0.87 ± 0.13 | 5.0 | 3.6 ± 0.5 |
| 25 | 1.6 | 1.8 ± 0.5 | 12.5 | 8.1 ± 1.5 |
| 50 | 3.1 | 4.8 ± 1.3 | 25 | 27.0 ± 6.4 |
| 100 | 6.3 | 5.0 ± 1.2 | 50 | 32.7 ± 6.8 |
| 200 | 12.6 | 7.7 ± 1.6 | 100 | 52.5 ± 9.2 |

Toxicokinetics

| | Cₘₐₓ (μM) | AUC (μM · h) |
|---|---|---|
| No Effect Dose | | |
| Rat: 15 mg/kg BID (28 d) | 17 | 70 |
| Dog (M): 15 mg/kg BID (12 m) | 74 | 401 |
| Effect Dose | | |
| Rat: 45 mg/kg BID (28 d) | 44 | 161 |
| Dog (F): 15 mg/kg BID (12 m) | 69 | 431 |

Oral administration of single ascending doses of β-D-D4FC resulted in a dose dependent increase in the mean Cₘₐₓ and AUC values. A Cₘₐₓ of 2.5 μM was reached following a 50 mg dose with either the buffered solution or the tablet. At the 200 mg dose the mean Cₘₐₓ remains above 5 μM for >3.5 hours. Therefore, β-D-D4FC has high oral bioavailability.

The mean terminal half-life in plasma (t_{1/2}) of β-D-D4FC when delivered as a buffered solution (10, 25 & 50 mg) ranged from 6.8±4.2 h to 15.6±12.4 h and the mean Tₘₐₓ for all dose levels with the buffered solutions was 1.1±0.2 hours.

The mean t_{1/2} of β-D-D4FC when delivered as an enteric-coated tablet (50, 100 and 200 mg) ranged from 11.3±1.6 hours to 14.3±3.1 hours and the mean Tₘₐₓ ranged from 1.96±0.57 hours to 2.9±1.6 hours.

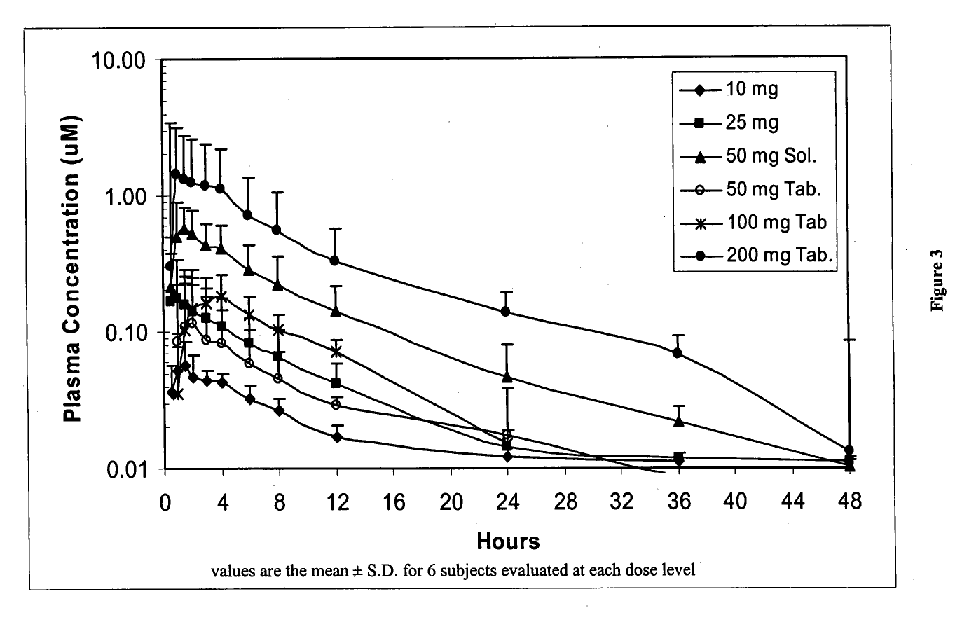

The mean concentration of 5FC following dosing with the buffered solution was 15±7% of that for β-D-D4FC, compared to only 2.5±2.5% of the β-D-D4FC concentration following dosing with the tablet formulation. Thus, there was approximately a 3-fold decrease in 5FC with enteric-coated tablets. See FIG. 3. The enteric coated tablets significantly reduce the level of 5FC in plasma compared to the buffered solution. No 5-fluorouracil was detected in the plasma at doses as high as 200 mg.

Single doses of β-D-D4FC were well tolerated and no serious adverse events were observed at any dose level. All adverse events were mild and occurred no more frequently than with placebo. Headache and fatigue were the most common.

Based on the in vitro potency of β-D-D4FC against both wild type and NRTI-resistant HIV-1, AUC values associated with a peak plasma concentration of 2.5 μM are anticipated to be sufficient to inhibit >80% of the clinically significant HIV strains. This single dose study demonstrated that the desired plasma concentrations of β-D-D4FC are easily achieved with a dose of 50 mg. The amount of 5FC produced was markedly reduced using enteric-coated β-D-D4FC tablets. The data suggests that β-D-D4FC could be useful as a once-a-day component of treatment regimens for NRTI-experienced patients.

### Example 10

Single Dose β-D-D4FC—48 Hour Study

β-D-D4FC was administered as a single oral dose to HIV-1 infected males at doses of 10, 25 or 50 mg as buffered solutions or 50, 100 or 200 mg as enteric-coated tablets. Blood samples, obtained over a 48-hour period for pharmacokinetic analysis, were analyzed for β-D-D4FC and its metabolites, 5-fluorocytidine, (5FC) and 5-fluorouracil (5FU), using LC/MS/MS. HIV RNA levels were also determined using quantitative real-time RT-PCR.

The study consisted of a double-blind, randomized, placebo-controlled, three-period crossover, dose escalation design in HIV-infected male subjects. Five dose levels (10, 25, 50, 100, and 200 mg) were administered in three cohort studies. Seven days elapsed between the end of one cohort and the start of the next dosing period.

TABLE 12
Subject Demographics

| Category | |
|---|---|
| Planned/entered/discontinued | 18/18/0 |
| Mean Age in Years | 39.1 |
| (Range) | (24-51) |
| Male/Female | 18/0 |
| Race | |
| Caucasian | 16 |
| Black | 2 |

In Cohort 1, subjects received either 10 mg or 25 mg of β-D-D4FC or placebo as a buffered solution. In Cohort 2, subjects received either placebo or 50 mg of β-D-D4FC as coated tablets, or as a buffered solution, to determine the bioavailability of coated tablets versus buffered solution. In Cohort 3, subjects received either 100 mg, or 200 mg, or placebo.

| | |
|---|---|
| Dosing | |
| Period | Dose |

COHORT 1

| | |
|---|---|
| 1 | 10 mg (N = 4) or placebo (N = 2) |
| 2 | 10 mg (N = 2), 25 mg (N = 2) or |
| | placebo (N = 2) |
| 3 | 25 mg (N = 4) or placebo (N = 2) |

COHORT 2

| | |
|---|---|
| 1 | 50 mg BSF* (N = 4) or placebo (N = 2) |
| 2 | 50 mg BSF (N = 2), 50 mg EC**(N = 2) or |
| | placebo (N = 2) |
| 3 | 50 mg EC (N = 4) or placebo (N = 2) |

COHORT 3

| | |
|---|---|
| 1 | 100 mg (N = 4) or placebo (N = 2) |
| 2 | 100 mg (N = 2), 200 mg (N = 2) or |
| | placebo (N = 2) |
| 3 | 200 mg (N = 4) or placebo (N = 2) |

*BSF = buffered solution formulation,
**EC = enteric coated tablet

Samples as part of a full-profile plasma PK were obtained over a 48 hour period following a single oral dose of β-D-D4FC. Plasma was analyzed for the presence of β-D-D4FC, 5FC, and 5FU using LC/MS/MS. In addition, cryopreserved sodium EDTA plasma samples (0.05 mL) collected 0, 12, 24, and 48 hours post dose were analyzed for HIV-1 RNA levels in quadruplicate. Plasma HIV-1 RNA levels were measured by quantitative reverse transcriptase-polymerase chain reaction (Q-RT-PCR) essentially as described by Stuyver, L. J. et al., “Antiviral activity and cellular toxicity of modified 2′,3′-dideoxy-2′,3′-didehydrocytidine analogues” Antimicrob Agents Chemother. 2002, 46, 3854-3860.

Viral RNA from the first and last plasma samples from each period (P1-0 and P1-48; P2-0 and P2-48; and P3-0 and P3-48) for each patient were amplified, sequenced and analyzed for NRTI mutations (M41, K65, K70, L74, V75, M184, T215), NNRTI mutations (L100, K103, V108, Y181), and multi-drug resistance mutations (T69, Q151). Sequence interpretation was done using TRUGENE HIV-1 software (Bayer NAD, Suwanee, Ga.).

After a single oral dose of β-D-D4FC, viral loads dropped significantly over 48 hours with an average reduction of 0.4±0.2 log₁₀ for all dose levels tested. The antiviral response over the 48 hour period was not dose dependent, potentially due to the long intracellular half-life of β-D-D4FC-TP and the short observation period. At the 10 mg dose, a 0.42±0.2 log₁₀ was observed (p=0.005), while at 100 mg a similar effect of 0.44±0.17 was noted (P=0.0007). The dose-independent significant antiviral response was also observed at the 24 hour time point, with an average reduction of 0.11±0.11 (p=0.03). The 12 hour time point was not significantly different from baseline.

TABLE 13
Viral Load Decline with β-D-D4FC & Competing Nucleosides

| | | | | | Viral Load |
|---|---|---|---|---|---|
| | | | | Duration of | Decline |
| Treatment | Company | Dose (mg) | Schedule | Dosing | (log) |
| β-D-D4FC | PSL | 50 | QD | 10 | −1.67 |
| SPD 754 | SHP | 1200 | QD | 10 | −1.62 |
| FTC | GILD/σ | 200 | QD | 12 | −1.7 |
| TFV | GILD | 300 | QD | 28 | −1.57 |
| RCV¹ | PSL | 200 to 600 | QD | 14 | −2.02 to −2.43 |
| FLT² | BI | 7.5 | QD | 28 | −1.88 |
| L-d4FC^{2,3} | ACH | 50 | QD | 21 | −1.69 |
| DAPD² | GILD/σ | 300 | BD | 14 | −1.5 |
| 3TC | GSK | 150 | BD | 12 | −1.45 |
| ABC | GSK | 600 | BD | 7 | −1.00 |
| AZT | GSK | 300 | BD | 14 | −0.6 |

¹Not monotherapy, in combination with EFV & D4T;
²Add-on study conducted in experienced patients;
³Mean of 7 of 18 patients; CD4 declined

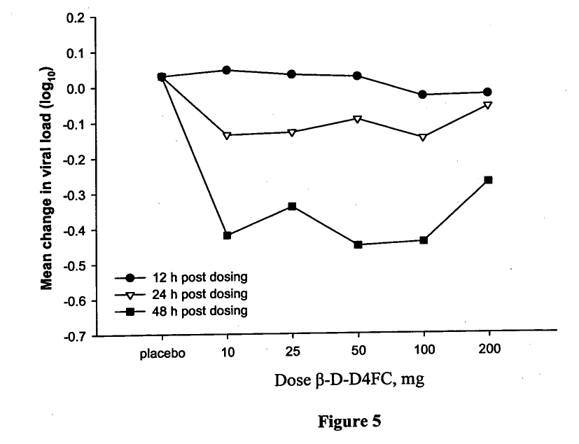

The mean change in viral load (in log₁₀) was compared to the administered dose of β-D-D4FC (FIG. 5). A significant decrease in plasma HIV-1 RNA was observed after a single 10 mg dose of β-D-D4FC. The most consistent decreases were observed 48 hours after treatment, when a mean reduction of 0.42 log₁₀ copies/mL was reached. A smaller but statistically significant reduction (p=0.03) was also observed 24 hours after administration. The 12 hour time-point was not significantly different from baseline. Increasing the dose from 10 mg up to 200 mg did not result in a more pronounced antiviral effect after 48 hours.

The mean plasma Cₘₐₓ values for single oral doses of β-D-D4FC were dose dependent. The Cₘₐₓ ranged from 1 to 8 μM. A maximal effect of viral inhibition was obtained at the lowest Cₘₐₓ (0.87 μM), which is equivalent to the in vitro EC₉₀ value for wild type virus. All available viral strains (n=36) were sequenced in the reverse transcriptase gene before (n=18 strains) and after (n=18 strains) the treatment schedule. Wild type viral genotype was found in all but one subject who showed the following genotype at baseline: L41+N103+C181+W210+D215, suggesting past exposure to AZT (possibly in another host) and non-nucleoside analogues. This subject received the 10 mg, placebo, and 25 mg treatment schedule, and showed a 0.61, −0.05, and 0.43 log₁₀ drop in viral load, respectively. The one subject infected with a mutant virus responded as well as those subjects infected with wild type virus, demonstrating β-D-D4FC's effectiveness against drug resistant viral strains. The viral genotype for all subjects remained unchanged at the end of the treatment schedule.

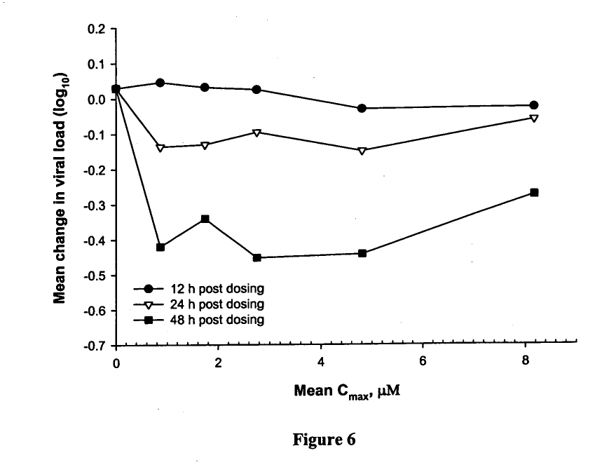

The relationship between antiviral effect and the amount of compound exposure was evaluated by plotting viral load versus mean Cₘₐₓ values (FIG. 6). Maximal reduction in viral load occurred at 48 hours post administration. A maximal effect of viral inhibition was obtained at the lowest Cₘₐₓ (0.87±0.15 μM), which is equivalent to the in vitro EC₉₀ value for wild type virus (Schinazi, R. F. et al., “DPC 817: a cytidine nucleoside analog with activity against zidovudine- and lamivudine-resistant viral variants” Antimicrob Agents Chemother. 2002, 46, 1394-1401).

The mean Tₘₐₓ with the buffered solution was 1.1±0.2 hours, while the mean Tₘₐₓ with the enteric-coated tablets was 2.9±1.6 hours. The mean plasma half-life (t_{1/2}) was 12.3±4.0 hours. Low levels of 5FC were detected in plasma. Dosing with enteric-coated tablets reduced the amount of 5FC in the plasma more than three-fold compared to the buffered solution.

The relationship between the Cₘₐₓ and the AUC values was studied for each dose group [10, 25, and 50 mg (buffered solutions), 50, 100, and 200 mg (enteric-coated tablets); 6 subjects per group], and a linear relationship between Cₘₐₓ and AUC was found. AUC and Cₘₐₓ values were dose dependent.

A summary of the changes in HIV-1 RNA plasma levels that occurred after single dose administration of β-D-D4FC is given in Table 14.

TABLE 14
Changes in Plasma HIV-1 RNA After Single Dose Administration of β-D-D4FC

| time, h | placebo | 10 mg | 25 mg | 50s mg | 50t mg | 100 mg | 200 mg |
|---|---|---|---|---|---|---|---|
| 0 | 0.00 ± 0.00 | 0.00 ± 0.00 | 0.00 ± 0.00 | 0.00 ± 0.00 | 0.00 ± 0.00 | 0.00 ± 0.00 | 0.00 ± 0.00 |
| 12 | 0.02 ± 0.23 | 0.05 ± 0.09 | 0.03 ± 0.09 | 0.00 ± 0.00 | 0.05 ± 0.07 | −0.03 ± 0.08 | −0.02 ± 0.24 |
| 24 | 0.08 ± 0.16 | −0.12 ± 0.14 | −0.13 ± 0.10 | −0.11 ± 0.07 | −0.08 ± 0.07 | −0.15 ± 0.14 | −0.06 ± 0.22 |
| 48 | 0.03 ± 0.12 | −0.42 ± 0.20 | −0.34 ± 0.26 | −0.44 ± 0.02 | −0.46 ± 0.25 | −0.44 ± 0.17 | −0.27 ± 0.28 |
| p-value for 24 hr | | 0.03 | 0.006 | 0.01 | 0.01 | 0.007 | 0.16 |
| p-value for 48 hr | | 0.005 | 0.03 | <0.00001 | 0.07 | 0.0007 | 0.05 |

The Cₘₐₓ (range =1-8 μM) values at all doses were greater than the in vitro EC₉₀ for wild type virus (EC₉₀=0.9 μM). The long plasma half-life of the nucleoside (12.3±4.0 h) and the long intracellular half-life of the triphosphate (13-17 h) suggests a once a day dose for β-D-D4FC (Schinazi, R. F. et al., “DPC 817: a cytidine nucleoside analog with activity against zidovudine- and lamivudine-resistant viral variants” Antimicrob Agents Chemother. 2002, 46, 1394-1401). There was a statistically significant reduction in mean plasma viral load of 0.42±0.2 log₁₀ after a single dose administration of β-D-D4FC (p<0.03). Viral load reductions were not dose dependent. Similar viral load reductions were observed after single dose administration of 25 to 200 mg of β-D-D4FC. A viral load reduction was observed at the lowest compound concentration tested (10 mg dose or ≈1 μM as Cₘₐₓ). An in vitro concentration of <1 μM is sufficient to reduce viral replication by 90%.

Again, the enteric-coated tablets significantly reduced the level of 5FC in the plasma compared to the buffered solution. No 5FU was observed in the plasma at doses as high as 200 mg (single dose).

Further, no resistance related changes were detected throughout any of the β-D-D4FC treatment schedules, i.e., P3-48 viral strains were identical to P1-0 viral strains.

All subjects, except Subject 106, had wild type sequences at P1-0. Only Subject 106 was infected with a viral strain harboring resistance mutations. Subject 106, at baseline, had the following detected mutations: L41+N103+C181+W210+D215 in a HIV-1 genotype B backbone. The D215 mutation has been associated with past exposure to AZT (de Ronde, A. et al., “Establishment of new transmissible and drug-sensitive human immunodeficiency virus type 1 wild types due to transmission of nucleoside analogue-resistant virus” J. Virol. 2001, 75, 595-602).

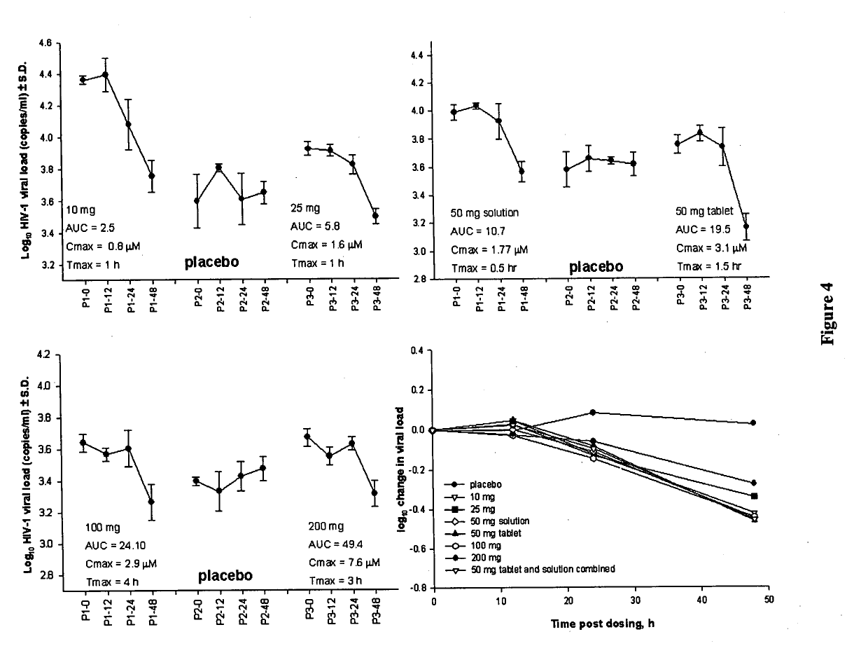

The viral load reductions in Subject 106 were not significantly different from viral load profiles seen in other subjects within Cohort 1, or in any other cohort. The viral load drop after single 10 mg and 25 mg dose administration is shown in FIG. 4A. Observed reductions in viral load after single dose administration of 10 or 25 mg of β-D-D4FC in Subject 106 were similar to those observed in the other subjects infected with wild type virus. The interpretation of this genotypic resistance pattern is provided in Table 15.

TABLE 15
Mutation Resistance Pattern Interpretation
Relevant RT Mutations: M41L A98S K103N Y181C L210W

| | Resistance Interpretation |
|---|---|
| Nucleoside RT Inhibitors | |
| Zidovudine | Possible Resistance |
| Didanosine | No Evidence of Resistance |
| Zalcitabine | No Evidence of Resistance |
| Lamivudine | No Evidence of Resistance |
| Stavudine | No Evidence of Resistance |
| Abacavir | Possible Resistance |
| Tenofovir | Insufficient Evidence |
| Foscarnet | Insufficient Evidence |
| Non-Nucleoside RT Inhibitors | |
| Nevirapine | Resistance |
| Delavirdine | Resistance |
| Efavirenz | Resistance |

The data suggests that β-D-D4FC maintained its antiviral potency against viral strains with the indicated resistance pattern.

Single doses of β-D-D4FC were well tolerated at all doses tested and no serious adverse events were observed at any dose level. All adverse events were mild in nature—fatigue and headache were the most commonly reported adverse events. All adverse events occurred no more frequently than with placebo. There was no relationship between dose and the number or intensity of the adverse events.

Based on the in vitro potency of β-D-D4FC against both wild type and NRTI-resistant HIV-1, the favorable PK values, as well as the in vivo activity observed after only a single dose of drug, β-D-D4FC can be useful as a once-a-day component of treatment regimens for NRTI-experienced and treatment-naïve patients.

### Example 11

Effect of Food on the Pharmacokinetics of β-D-D4FC.

The effect of food on the pharmacokinetics of β-D-D4FC was assessed in a fed versus fasted 2×2 crossover with 6 subjects in Cohort 4. The subjects received a single dose of 100 mg β-D-D4FC coated tablets either in a fasted state or after a high fat standard meal (FDA).

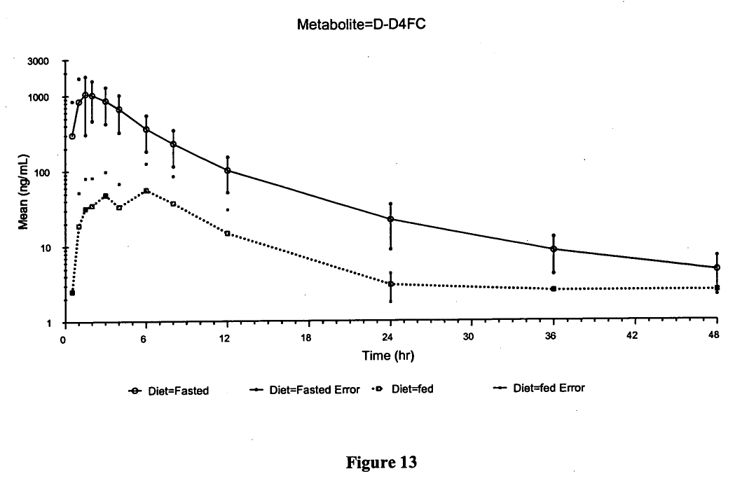

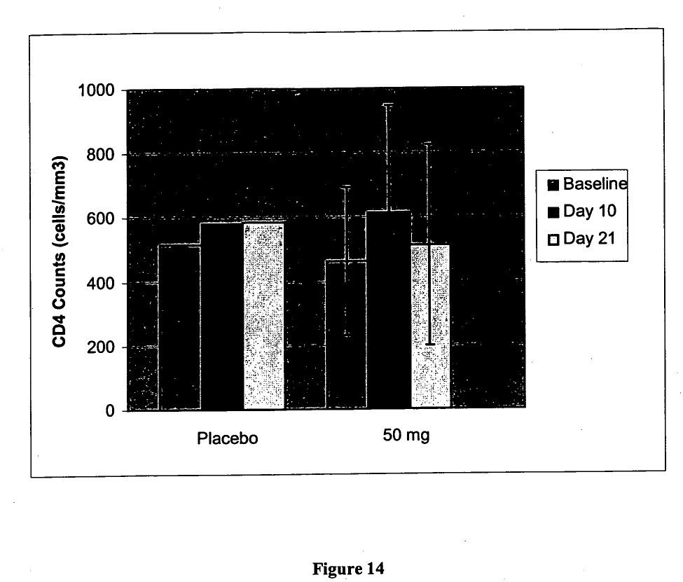

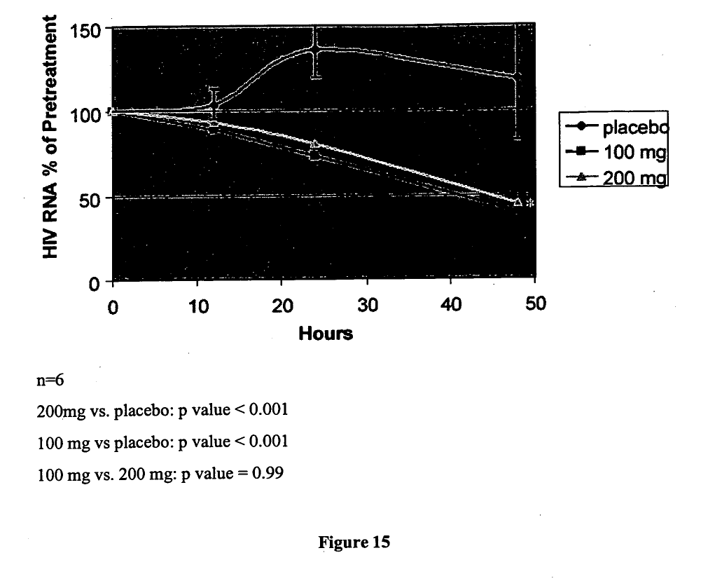

The results for all plasma pharmacokinetic variables are summarized in Tables 16 and 17, below. FIG. 13 shows plasma concentration over time for the fed and fasted regimen. The calculated data clearly indicate a food effect: β-D-D4FC Cₘₐₓ and AUC values in the “fed” regimen are less than 1/10 of the Cₘₐₓ and AUC values in the “fasted” regimen. In addition, the high fat meal resulted in 5-FC values 10-fold higher in the “fed” regimen than in the “fasted” regimen.

TABLE 16
D-D4FC

| | Dose | | | | | | | |
|---|---|---|---|---|---|---|---|---|
| | PART A | | | | | | PART B | |
| Parameter | 10 MG | 25 MG | 50 MG | 50 MG | 100 MG | 200 MG | 100 MG | 100 MG |
| [unit] | SOLUTION | SOLUTION | SOLUTION | TABLET | TABLET | TABLET | FASTED | FED |
| Cₘₐₓ [ng/mL] | 197 ± | 406 ± | 668 ± | 572 ± | 1,125 ± | 1,749 ± | 1,159 ± | 90 ± |
| | 32 | 110 | 155 | 232 | 264 | 358 | 732 | 71 |
| tmax [h] | 1.00 ± | 1.17 ± | 1.00 ± | 2.00 ± | 2.92 ± | 3.33 ± | 1.83 ± | 2.50 ± |
| | 0.00 | 0.93 | 0.32 | 0.84 | 1.02 | 1.63 | 0.68 | 2.00 |
| AUC(0-τ) [ng · h/mL] | 843 ± | 1,826 ± | 3,470 ± | 3,196 ± | 7,014 ± | 11,185 ± | 6,112 ± | 542 ± |
| | 93 | 267 | 609 | 1,125 | 1,436 | 1,971 | 3,424 | 381 |
| AUC(0-∞) [ng · h/mL] | 858 ± | 1,842 ± | 3,498 ± | 3,221 ± | 7,100 ± | 11,373 ± | 6,170 ± | 643 ± |
| | 91 | 269 | 611 | 1,128 | 1,476 | 1,992 | 3,455 | 363 |
| t_{1/2} λz [h] | 4.20 ± | 4.49 ± | 7.73 ± | 7.02 ± | 9.25 ± | 10.92 ± | 8.61 ± | 4.38 ± |
| | 0.50 | 1.20 | 2.46 | 1.77 | 2.25 | 2.53 | 1.54 | 1.84 |
| CLₜₒₜ/F [mL/h] | 11,765 ± | 13,812 ± | 14,715 ± | 17,492 ± | 14,545 ± | 18,079 ± | 28,669 ± | 202,649 ± |
| | 1,293 | 1,990 | 2,958 | 6,987 | 2,726 | 3,462 | 32,704 | 233,716 |
| CLᵣ [L/h] | 8.15 ± | 7.20 ± | 4.39 ± | 4.30 ± | 7.46 ± | 7.48 ± | — | — |
| | 1.91 | 3.27 | 2.32 | 1.41 | 1.41 | 0.98 | | |

Altogether, β-D-D4FC was absorbed fairly well from tablets and solution. Considering easier dosing, and the appearance of less metabolite production with the coated tablet, the preferred form for administration of β-D-D4FC is the enteric-coated tablet.

TABLE 17
5-FC

| | Dose | | | | | | | |
|---|---|---|---|---|---|---|---|---|
| | PART A | | | | | | PART B | |
| Parameter | 10 MG | 25 MG | 50 MG | 50 MG | 100 MG | 200 MG | 100 MG | 100 MG |
| [unit] | SOLUTION | SOLUTION | SOLUTION | TABLET | TABLET | TABLET | FASTED | FED |
| Cₘₐₓ [ng/mL] | 9.16 ± | 26.69 ± | 67 ± | 29 ± | 25 ± | 271 ± | 66 ± | 604 ± |
| | 3.98 | 24.33 | 49 | 41 | 14 | 385 | 99 | 222 |
| tmax[h] | 1.08 ± | 1.33 ± | 1.33 ± | 1.58 ± | 3.67 ± | 3.33 ± | 6.92 ± | 4.50 ± |
| | 0.38 | 0.88 | 0.26 | 0.97 | 0.82 | 3.19 | 8.73 | 1.23 |
| AUC(0-τ) | 136 ± | 234 ± | 491 ± | 333 ± | 309 ± | 1,665 ± | 511 ± | 4,948 ± |
| [ng · h/mL] | 14 | 108 | 257 | 369 | 80 | 1,527 | 418 | 903 |
| AUC(0-∞) | 149 ± | 250 ± | 512 ± | 465 ± | 340 ± | 1,887 ± | 588 ± | 5,017 ± |
| [ng · h/mL] | 16 | 112 | 256 | 424 | 78 | 1,661 | 387 | 813 |
| t_{1/2} λz [h] | 2.41 ± | 4.60 ± | 5.93 ± | 7.06 ± | 8.50 ± | 10.48 ± | 17.02 ± | 5.41 ± |
| | 1.38 | 1.96 | 1.48 | 4.26 | 2.79 | 2.92 | 12.15 | 0.67 |
| CLₜₒₜ/F | 67 957 ± | 115 262 ± | 127 303 ± | 180 143 ± | 307 932 ± | 169 692 ± | 216 764 ± | 20 385 ± |
| [mL/h] | 7,501 | 44 362 | 77 441 | 121 440 | 71 219 | 104 014 | 97 628 | 3 409 |
| CLr [L/h] | 1.39 ± | 1.27 ± | 1.24 ± | 1.17 ± | 10.26 ± | 4.60 ± | — | — |
| | 0.18 | 0.09 | 0.10 | 0.05 | 6.17 | 1.12 | | |

The maximum concentrations Cₘₐₓ of 5-FC in plasma ranged between 9.16±3.98 mg/mL in the 10 mg dosing period to 271±385 mg/mL in the 200 mg dosing period for study drug intake in a fasted state. In the 100 mg “fed” dosing period in cohort 4,5-FC Cₘₐₓ was distinctly higher, with 604±222 ng/mL.

The Area Under the Curve (AUC_{(0-τ)}) ranged from 136±14 ng*h/mL in the 10 mg dosing period to 4,948±903 ng*h/mL in the 100 mg “fed” dosing period. The time to reach maximum concentration (tₘₐₓ) in Cohorts 1-3 was comparable to tₘₐₓ for β-D-D4FC and increased with dose. The tₘₐₓ values in Cohort 4 (100 mg “fed” and “fasted”) were considerably higher than for Cohorts 1-3. In contrast to the parent compound, tₘₐₓ values showed no relevant differences between coated tablet and buffered solution formulation.

Less 5-FC was generated with coated tablets than with buffered solution formulation. Furthermore, the 5-FC concentrations in the “fed” regimen were about 10 times higher than in the “fasted” regimen in Cohort 4.

### Example 12

Effect of Food on the Pharmacokinetics of β-D-D4FC.—Dry Compression (DC) Versus Coated Pellet (Bead)

The effect of food on the pharmacokinetics of β-D-D4FC was assessed in 24 human subjects based on a wet granulation formulation or a coated pellet formulation. The subjects received a single dose of 100 mg β-D-D4FC coated tablets either in a feed or fasted state. The plasma pharmacokinetic variables were measured and are tabulated in Tables 18 and 19.

TABLE 18
Pharmacokinetics of Dry Compressed (DC) Tablet

| | | | | Lower limit | Upper limit |
|---|---|---|---|---|---|
| | | | Geometric | of 90% | of 90% |
| | 100 mg | 100 mg | Mean Ratio | Confidence | Confidence |
| Parameter | “fasted” | “fed” | (fasted/fed) | Interval | Interval |
| Cₘₐₓ [ng/mL] | 917 | 47 | 19.5811 | 2.40708 | 159.289 |
| AUC(0-τ) | 4938 | 410 | 12.0439 | 2.83241 | 51.213 |
| [ng · h/mL] | | | | | |
| AUC(0-∞) | 4993 | 540 | 9.4831 | 1.65958 | 54.189 |
| [ng · h/mL] | | | | | |

TABLE 19
Pellet (bead) formulation and Dry Compressed (DC) tablet

| | Tₘₐₓ | Cₘₐₓ | AUC | Cₘₐₓ ratio | AUC ratio |
|---|---|---|---|---|---|
| PK | (h) | (nM) | (nM · h) | (%) | (%) |
| BEADS −1 h | 1.9 ± 0.6 | 4.0 ± 1.9 | 20.2 ± 8.3 | 164 ± 205^{@} | 153 ± 148^{@} |
| FASTED | | | | | |
| BEADS 0 h | 3.8 ± 2.5 | 2.6 ± 2.1 | 15.8 ± 11 | 68 ± 47^{#} | 84 ± 54^{#} |
| WITH | | | | | |
| FOOD | | | | | |
| BEADS +2 h | 4.5 ± 1.5 | 2.6 ± 2.1 | 17.7 ± 10.7 | 74 ± 49^{#} | 100 ± 71^{#} |
| AFTER | | | | | |
| MEAL | | | | | |
| DC −1 h | 2.5 ± 2.4 | 4.1 ± 2.6 | 19.6 ± 11.8 | | |
| FASTED | | | | | |

^{@}compared to direct compression (DC) Tablet;
^{#}compared to Beads −1 h

This Example demonstrates that certain formulations of β-D-D4FC provide improved pharmacokinetic parameters when administered with food or 2 hours after food compared to standard dry compressed (DC) tablets of β-D-D4FC administered with food.

Although the present invention has been described with respect to a specific embodiment, the details of the embodiment is not to be construed as limitations. Various equivalents, changes and modification may be made without departing from the spirit and the scope of this invention, and it is understood that such equivalent embodiments are part of this invention. The present invention may be embodied in other specific forms without departing from the spirit or essential attributes thereof and, accordingly, reference should be made to the appended claims as further indicating the scope of the invention.

1. A pharmaceutical composition comprising β-D-2′,3′-didehydro-2′,3′-dideoxy-5-fluorocytidine (β-D-D4FC) that is enterically coated.

2. The pharmaceutical composition of claim 1, wherein the composition is in a once a day dosage form.

3. The pharmaceutical composition of claim 1, wherein the composition is in the form of a tablet.

4. The pharmaceutical composition of claim 1, wherein the composition is in the form of a capsule.

5. The pharmaceutical composition of claim 1, wherein the enteric coating is in the form of enteric coating on beads.

6. The pharmaceutical composition of claim 5, wherein the composition is in the form of beadlets in a capsule.

7. A pharmaceutical composition comprising:
a. a core comprising β-D-D4FC, optionally pharmaceutically acceptable excipients,
b. an optional separating layer;
c. an enteric layer, optionally with a pharmaceutically acceptable excipient; and
d. an optional finishing layer.

8. The pharmaceutical composition of claim 7, wherein the β-D-D4FC is optionally layered on a seed/sphere.

9. The pharmaceutical composition of claim 8, wherein the seed is a water insoluble seed.

10. The pharmaceutical composition of claim 9, wherein the water insoluble seed is an oxide, cellulose, organic polymer, or mixture thereof.

11. The pharmaceutical composition of claim 8, wherein the seed is a water soluble seed.

12. The pharmaceutical composition of claim 11, wherein the water soluble seed is an inorganic salt, sugar, non-pareil, or mixture thereof.

13. The pharmaceutical composition of claim 7, wherein the enteric layer is made from one or more layers of fatty acids, stearic acid, palmitic acid, wax, shellac, phthalate, cellulose acetate phthalate, poly(vinyl acetate) phthalate-based Sureteric, latex of cellulose acetophtalate (CAP), Aquateric, acrylate, acrylic resin, copolymers of acrylic acids and acrylates, copolymers of methacrylic acid and ethyl acrylate, Eudragit L30D, Eudragit L30D-55, Eudragit L100-55, Eudragit FS 30 D, Instacoat EN-Sol, Instacoat EN-HPMC-P, Instacoat EN Super, Instacoat EN II, Acryl-Eze, or a combination thereof.

14. The pharmaceutical composition of claim 7, wherein the enteric layer is made from one or more layers phthalate, acrylate, copolymers of acrylic acids and acrylates, copolymers of methacrylic acid and ethyl acrylate, or a combination thereof.

15. The pharmaceutical composition of claim 7, wherein the enteric layer is made from Eudragit or Acryl-Eze.

16. The pharmaceutical composition of claim 7, wherein the composition further comprises pharmaceutically acceptable plasticizers.

17. The pharmaceutical composition of claim 16, wherein the plasticizer is selected from the group consisting of triethylcitrate (Citroflex-2), tributylcitrate (Citroflex-4), acetyltributylcitrate (Citroflex-A4), dibutyl sebacate (DBS), diethylphtalate (DEP), acetylated monoglyceride (Myvacet 9-40), polyethylenoglycols and 1,2-propylene glycol.

18. The pharmaceutical composition of claim 16, wherein the plasticizer is triethylcitrate.

19. The pharmaceutical composition of claim 7, wherein the composition further comprises pharmaceutically acceptable lubricant.

20. The pharmaceutical composition of claim 19, wherein the lubricant is talc, stearic acid, stearate, such as magnesium stearate, sodium stearyl fumarate, glyceryl behenate, kaolin, aerosol, or colloidal silicon dioxide.

21. The pharmaceutical composition of claim 19, wherein the lubricant is talc or colloidal silicon dioxide.

22. The pharmaceutical composition of claim 7, wherein the composition further comprises pharmaceutically acceptable excipient.

23. The pharmaceutical composition of claim 22, wherein the excipient is lactose, starches, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin or castor oil.

24. The pharmaceutical composition of claim 22, wherein the excipient is sodium carboxymethyl cellulose, magnesium stearate, micro-crystalline cellulose, triethyl citrate, or sodium phosphate.

25. The pharmaceutical composition of claim 7, wherein the composition further comprises pharmaceutically acceptable adhesive.

26. The pharmaceutical composition of claim 25, wherein the adhesive is polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), or xanthan gum.

27. The pharmaceutical composition of claim 25, wherein the adhesive is polyvinyl pyrrolidone (PVP), hydroxypropyl methyl cellulose (HPMC), hydroxypropyl cellulse (HPC), or cross-linked carboxymethyl cellulose.

28. The pharmaceutical composition of claim 7, wherein the composition further comprises pharmaceutically acceptable diluent.

29. The pharmaceutical composition of claim 28, wherein the diluent is lactose, starch, mannitol, sodium carboxymethyl cellulose, sodium starch glycolate, sodium chloride, potassium chloride, pigments, salts of alginic acid, talc, titanium dioxide, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin or castor oil.

30. The pharmaceutical composition of claim 25, wherein the diluent is mannitol or sodium carboxymethyl cellulose.

31. The pharmaceutical composition of claim 7, wherein the composition further comprises anti-foaming agent.

32. The pharmaceutical composition of claim 31, wherein the anti-foaming agent is a silicone.

33. The pharmaceutical composition of claim 31, wherein the anti-foaming agent is a simethicone.

34. A pharmaceutical composition comprising:
a. a core comprising:
β-D-D4FC,
alkaline buffer,
excipient and/or adhesive and/or lubricant, and
b. a separating layer comprising:
pH buffering substance
c. an enteric layer comprising:
enteric polymer, and
anti-foaming agent.

35. The pharmaceutical composition of claim 22, wherein the alkaline buffer is sodium bicarbonate or sodium diphosphate.

36. The pharmaceutical composition of claim 22, wherein the excipient and/or adhesive and/or lubricant are selected from the group consisting of microcrystalline cellulose, crospovidone, magnesium stearate, Prosolv, mannitol, hydroxypropyl cellulose, or croscarmellose.

37. The pharmaceutical composition of claim 22, wherein the pH buffering substance is titanium dioxide or HPMC.

38. The pharmaceutical composition of claim 22, wherein the anti-foaming agent is a simethicone.

39. The pharmaceutical composition of claim 22, wherein enteric polymer is a phthalate or acrylate polymer or a copolymer of acrylate and acrylic acid.

40. The pharmaceutical composition of claim 22, comprising:
a. a core comprising:
β-D-D4FC,
Sodium Bicarbonate,
Microcrystalline Cellulose,
Crospovidone, and
Magnesium Stearate, and
b. a separating layer comprising:
titanium dioxide
c. an enteric layer comprising:
Sureteric, and
Simethicone.

41. The pharmaceutical composition of claim 22, comprising:
a. a core comprising:
β-D-D4FC,
Sodium Diphosphate, Dibasic,
Prosolv,
Mannitol,
Hydroxypropyl Cellulose,
Croscarmellose Sodium, and
Magnesium Stearate, and
b. a separation layer comprising:
titanium dioxide
c. an enteric layer comprising:
Eudragit L30 D-55,
Triethyl Citrate,
Talc,
Simethicone, and
Sodium Hydroxide.

42. A pharmaceutical composition comprising:
a. a core comprising:
β-D-D4FC,
alkaline buffer,
excipient and/or adhesive and/or lubricant, and
an inert water-soluble seed,
b. a separating layer comprising:
pH buffering substance
c. an enteric layer comprising:
enteric polymer, and
anti-foaming agent, and
d. a finishing layer comprising:
hard gelatin capsule.

43. The pharmaceutical composition of claim 42, wherein the alkaline buffer is sodium bicarbonate or sodium diphosphate.

44. The pharmaceutical composition of claim 42, wherein the alkaline buffer is sodium bicarbonate or sodium diphosphate.

45. The pharmaceutical composition of claim 42, wherein the excipient and/or adhesive and/or lubricant are selected from the group consisting of microcrystalline cellulose, crospovidone, magnesium stearate, Prosolv, mannitol, hydroxypropyl cellulose, or croscarmellose.

46. The pharmaceutical composition of claim 42, wherein the pH buffering substance is titanium dioxide or HPMC.

47. The pharmaceutical composition of claim 42, wherein the anti-foaming agent is a simethicone.

48. The pharmaceutical composition of claim 42, wherein enteric polymer is a phthalate or acrylate polymer or a copolymer of acrylate and acrylic acid.

49. The pharmaceutical composition of claim 42, comprising:
a. a core comprising:
β-D-D4FC,
Sodium Diphosphate, Dibasic,
povidone
Magnesium Stearate, and
20/25 mesh Sugar Cores, and
b. a separation layer comprising:
HPMC E-5,
Simethicone, and
c. an enteric layer comprising:
Acryl-EZE MP
d. a finishing layer comprising:
gelatin capsule shells.

50. A formulation of β-D-D4FC wherein the Cₘₐₓ when administered to a human subject with food is about 50% to about 75% of Cₘₐₓ when administered to a human that has fasted.

51. The formulation of claim 50, wherein the Cₘₐₓ when administered to a human subject with food is about 50% of Cₘₐₓ when administered to a human that has fasted.

52. The formulation of claim 50, wherein the Cₘₐₓ when administered to a human subject with food is about 60% of Cₘₐₓ when administered to a human that has fasted.

53. The formulation of claim 50, wherein the Cₘₐₓ when administered to a human subject with food is about 70% of Cₘₐₓ when administered to a human that has fasted.

54. The formulation of claim 50, wherein the Cₘₐₓ when administered to a human subject with food is about 75% of Cₘₐₓ when administered to a human that has fasted.

55. A method for the treatment of HIV in a host, comprising administering to said host an effective treatment amount of a composition of claim 1.

56. The method of claim 55, wherein the host has fasted.

57. A method for the treatment of HIV in a host, comprising administering to said host an effective treatment amount of β-D-D4FC as a single oral dose per day such that the treatment amount achieves a plasma level of at least around 5 μM of β-D-D4FC.

58. The method of claim 57, wherein the single dose is from around 50 mg to around 200 mg of β-D-D4FC per day

59. The method of claim 57, wherein the single dose is 50 mg of β-D-D4FC per day.

60. The method of claim 57, wherein the single dose is 100 mg of β-D-D4FC per day.

61. The method of claim 57, wherein the HIV is wild-type HIV.

62. The method of claim 57, wherein the HIV is NRTI-resistant HIV.

63. The method of claim 62, wherein the NRTI-resistant HIV is NRTI-resistant HIV-1.

64. The method of claim 57, wherein the host is a treatment-naïve patient.

65. The method of claim 57, wherein the host is a NRTI-experienced patient.

66. A method for the treatment of HIV in a host, comprising administering to said host an effective treatment amount of β-D-D4FC as a single oral dose per day such that the treatment amount achieves a plasma level of at least around 5 μM of β-D-D4FC and wherein the β-D-D4FC is enterically coated.

67. The method of claim 66, wherein the host has fasted.

68. The method of claim 66, wherein the single dose is from around 50 mg to around 200 mg of β-D-D4FC per day

69. The method of claim 66, wherein the single dose is 50 mg of β-D-D4FC per day.

70. The method of claim 66, wherein the single dose is 100 mg of β-D-D4FC per day.

71. The method of claim 66, wherein the HIV is wild-type HIV.

72. The method of claim 66, wherein the HIV is NRTI-resistant HIV.

73. The method of claim 72, wherein the NRTI-resistant HIV is NRTI-resistant HIV-1.

74. The method of claim 66, wherein the host is a treatment-naïve patient.

75. The method of claim 66, wherein the host is a NRTI-experienced patient.

76. A method for the treatment of HIV in a host, comprising administering to said host an effective treatment amount of a pharmaceutical composition comprising:
a. a core comprising:
β-D-D4FC,
alkaline buffer,
excipient and/or adhesive and/or lubricant, and
b. a separating layer comprising:
pH buffering substance
c. an enteric layer comprising:
enteric polymer, and
anti-foaming agent.

77. The method of claim 76, wherein the alkaline buffer is sodium bicarbonate or sodium diphosphate.

78. The method of claim 76, wherein the excipient and/or adhesive and/or lubricant are selected from the group consisting of microcrystalline cellulose, crospovidone, magnesium stearate, Prosolv, mannitol, hydroxypropyl cellulose, or croscarmellose.

79. The method of claim 76, wherein the pH buffering substance is titanium dioxide or HPMC.

80. The method of claim 76, wherein the anti-foaming agent is a simethicone.

81. The method of claim 76, wherein enteric polymer is a phthalate or acrylate polymer or a copolymer of acrylate and acrylic acid.

82. The method of claim 76, comprising:
a. a core comprising:
β-D-D4FC,
Sodium Bicarbonate,
Microcrystalline Cellulose,
Crospovidone, and
Magnesium Stearate, and
b. a separating layer comprising:
titanium dioxide
c. an enteric layer comprising:
Sureteric, and
Simethicone.

83. The method of claim 76, comprising:
a. a core comprising:
β-D-D4FC,
Sodium Diphosphate, Dibasic,
Prosolv,
Mannitol,
Hydroxypropyl Cellulose,
Croscarmellose Sodium, and
Magnesium Stearate, and
b. a separation layer comprising:
titanium dioxide
c. an enteric layer comprising:
Eudragit L30 D-55,
Triethyl Citrate,
Talc,
Simethicone, and
Sodium Hydroxide.

84. A method for the treatment of HIV in a host, comprising administering to said host an effective treatment amount of a pharmaceutical composition comprising:
a. a core comprising:
β-D-D4FC,
alkaline buffer,
excipient and/or adhesive and/or lubricant, and
an inert water-soluble seed,
b. a separating layer comprising:
pH buffering substance
c. an enteric layer comprising:
enteric polymer, and
anti-foaming agent, and
d. a finishing layer comprising:
hard gelatin capsule.

85. The method of claim 84, wherein the alkaline buffer is sodium bicarbonate or sodium diphosphate.

86. The method of claim 84, wherein the alkaline buffer is sodium bicarbonate or sodium diphosphate.

87. The method of claim 84, wherein the excipient and/or adhesive and/or lubricant are selected from the group consisting of microcrystalline cellulose, crospovidone, magnesium stearate, Prosolv, mannitol, hydroxypropyl cellulose, or croscarmellose.

88. The method of claim 84, wherein the pH buffering substance is titanium dioxide or HPMC.

89. The method of claim 84, wherein the anti-foaming agent is a simethicone.

90. The method of claim 84, wherein enteric polymer is a phthalate or acrylate polymer or a copolymer of acrylate and acrylic acid.

91. The method of claim 84, comprising:
a. a core comprising:
β-D-D4FC,
Sodium Diphosphate, Dibasic,
povidone
Magnesium Stearate, and
20/25 mesh Sugar Cores, and
b. a separation layer comprising:
HPMC E-5,
Simethicone, and
c. an enteric layer comprising:
Acryl-EZE MP
d. a finishing layer comprising:
gelatin capsule shells.

92. The method of claim 55, wherein the host is a human.
